(19) United States
(12) Patent Application Publication
(10) Pub. No.: US 2026/0108539 A1
(43) Pub. Date: Apr. 23, 2026

(54) Cannabinoid Based Nanoplatform Composition and Methods for treating knee osteoarthritis

(71) Applicant: Cannabis Bioscience International Holdings Inc, Houston, TX (US)

(72) Inventors: Rosangel del Valle Andrades Fuentes, Caracas (VE); Nancy Paola Duarte Delgado, Bogotá (CO); Nancy Rodrigues Das Fontes, Viseu (PT); Stephanie Dona Hartmann, Caracas (VE); Ana Maria Maldonado Vacca, Bogotá (CO); Maria Angelica Clavijo Romero, Santa Marta (CO)

(73) Assignee: Cannabis Bioscience International Holdings Inc, Houston, TX (US)

(21) Appl. No.: 18/922,127

(22) Filed: Oct. 21, 2024

(51) Int. Cl. A61K 31/00; A61K 9/107; A61K 31/216; A61K 31/726; A61K 38/01; A61K 47/14

(52) U.S. Cl. CPC A61K 31/658; A61K 9/1075; A61K 31/216; A61K 31/726; A61K 38/014; A61K 47/14

(57) ABSTRACT

This document presents a novel composition and method for treating osteoarthritis using a cannabinoid-based nanoplatform administered via intra-articular injection. The composition includes phytocannabinoids such as CBG and CBC, along with hyaluronic acid, type II collagen, and calcium gluconate, embedded within a nanoplatform designed to enhance bioavailability and ensure controlled release in the synovial space. Tailored for osteoarthritis patients, the treatment alleviates joint pain, stiffness, reduced mobility, inflammation, sensations of friction, muscle weakness, and joint deformity. Cannabinoids act through multiple mechanisms, including interaction with CB1 and CB2 receptors, cytokine modulation, inhibition of NF-κB, and activation of the TRPV1 receptor, providing a potent anti-inflammatory effect. The formulation aims to halt cartilage degeneration, reduce pain, and slow osteoarthritis progression. The production process uses nanoemulsification techniques to create particles smaller than 200 nm, with pre-sterilization through membrane filtration, ensuring both efficacy and safety.

### FIELD OF THE DISCLOSURE

The technical field of the disclosure relates to medicinal, therapeutic, and healing Cannabis and cannabinoids, as well as pharmaceutical Cannabis and cannabinoids. The present disclosure relates to therapeutic compositions for the management of knee osteoarthritis, incorporating cannabinoids as active agents and designed for intra-articular injection. These compositions utilize advanced nanoplatforms to enhance bioavailability and therapeutic efficacy. This disclosure specifically addresses relief of persistent knee pain, limited morning stiffness, and reduced function. By combining cannabinoids with bioactive compounds such as cannabichromene (CBC), cannabigerol (CBG), hyaluronic acid, hydrolyzed collagen and calcium gluconate. The compositions offer a comprehensive approach to improving quality of life throughout the joint inflammation and degeneration of the knee joint.

### BACKGROUND INFORMATION

Osteoarthritis (OA) is a common joint disease affecting middle-aged to elderly individuals, with a higher incidence in women, particularly after age 50 (Palo et al., 2015). OA is a multifactorial condition involving mechanical forces, trauma, inflammation, biochemical reactions, and metabolic derangements (Ayhan, 2014). According to the World Health Organization (WHO), as of 2019, about 528 million people worldwide were living with OA, representing a 113% increase since 1990.

The knee is the most commonly affected joint, with 365 million cases, followed by the hip and hand. Many people experience moderate or severe symptoms requiring rehabilitation and in 2020, about 595 million individuals aged 30 and older were affected globally. Knee OA contributes significantly to disability and healthcare costs, as it is the leading reason for knee replacement surgeries (Steinmetz et al., 2023).

OA symptoms include persistent knee pain, stiffness, reduced function, and in more advanced cases, crepitus, restricted joint motion, and bone enlargement. Synovitis may occur, with inflammation of the connective tissue that lines the joints, produces synovial fluid, and lubricates and nourishes the articular cartilage causing pain at rest or at night. Bone remodeling plays a critical role in skeletal health, involving balanced activities of osteoblasts (OBs) and osteoclasts (OCs) to prevent bone loss and maintain structural integrity (Geng et al., 2023). Imbalance in these activities can lead to reduced bone mineral density and increased fragility, elevating the risk of fractures.

The endocannabinoid system (ECS) has been identified as a significant regulator of bone metabolism through the modulation of OB and OC activities. Cannabinoid receptor 2 (CB2R), expressed in immune cells and bone, modulates cytokine release to reduce inflammation and control bone resorption. Activation of CB2R in OCs regulates ion channels and intracellular calcium levels, while in OBs, CB2R promotes bone formation by enhancing Wnt/β-catenin signaling and key transcription factors CB2R modulation has been explored as a therapeutic target in inflammatory bone conditions, demonstrating potential in alleviating pain and bone loss (Geng et al., 2023). On the other hand, receptor type 1 (CB1R) is found in abundance in brain regions helping in-pain modulation.

Intra-articular injections with hyaluronic acid (HA) provide pain relief, restore synovial fluid properties, and promote cartilage health by enhancing matrix synthesis and inhibiting chondrocyte apoptosis. HA reduces inflammation and prevents cartilage erosion, while hydrolyzed collagen further promotes chondrocyte proliferation and extracellular matrix protein production (Jin et al., 2020). Furthermore, calcium gluconate helps prevent bone mass loss and treats declining bone density, making this combination a promising novel approach for managing knee OA symptoms and halting cartilage degeneration.

There is a clear need for a safer, more effective treatment option that addresses the symptoms of osteoarthritis without exposing patients to the risks associated with long-term use of conventional therapies like NSAIDs and corticosteroids. Current treatments fail to comprehensively target both inflammation and the underlying bone and cartilage degeneration in osteoarthritis. The present disclosure offers a novel cannabinoid-based composition using CBC and CBG to treat osteoarthritis through the endocannabinoid system. By modulating CB2 receptors, the formulation reduces inflammation, regulates bone remodeling, and preserves cartilage integrity. Additionally, the inclusion of hyaluronic acid and collagen enhances joint lubrication and promotes cartilage regeneration, while calcium gluconate contributes to the prevention of bone mass and density loss, offering critical support to the structural integrity of bones. This combination provides a novel and safer alternative to traditional therapies, specifically designed to alleviate pain, prevent cartilage breakdown, maintain bone health, and improve joint function in osteoarthritis patients.

### SUMMARY OF THE INVENTION

This disclosure presents a comprehensive approach to managing symptoms of knee osteoarthritis through three main embodiments: (1) a cannabinoid-based composition, (2) a method of treatment, and (3) a manufacturing method.

Composition: The main embodiment is a cannabinoid-based composition, wherein the composition comprising a) an active principle comprising at least two cannabinoid; b) an second active principle comprising at least one or more additional active agents including structural proteins, glycosaminoglycans, mineral supplement, or combinations thereof; and c) a stabilizing/solubilizing agent, wherein the composition is suitable for intra articular administration.

The composition is designed to reduce health risks associated with articular cartilage degeneration, while improving cartilage properties, synovial fluid viscosity, inhibit inflammation, and lubricate the joint, thereby improving joint function and providing comprehensive symptom relief.

Method of Treatment: This embodiment describes a therapeutic method particularly beneficial for patients with knee osteoarthritis. The treatment regimen includes quarterly intra-articular administration of the composition into the knee. This approach effectively relieves knee osteoarthritis symptoms including persistent knee pain, limited morning stiffness, and reduced function upon movement. By targeting the endocannabinoid system and utilizing nanoplatforms to enhance bioavailability, this treatment method optimizes symptom management and mitigates associated health risks such as chondrolysis, osteonecrosis, and fractures.

Manufacturing Method: The disclosure includes a method for producing the composition, which involves integrating cannabinoids, such as CBC, CBG, with natural supplements on nanoplatforms with previous procedures such as sonication and sterilization to improve therapeutic administration. The manufacturing method ensures consistent formulation and optimized bioavailability for oral and intra-articular administration routes, improving the efficacy and stability of the composition. The disclosure also includes a treatment designed for user convenience, containing: (a) intra-articular vial containing at least two cannabinoids, additional active agents; and (b) instructions for use.

This therapeutic approach is specifically formulated to address the clinical needs of patients suffering from knee osteoarthritis, focusing on alleviating symptoms such as joint pain, reduced knee mobility, and morning stiffness. The treatment leverages a multi-modal strategy that not only targets symptomatic relief but also mitigates the pathophysiological progression of the disease. In essence, this disclosure introduces an innovative, evidence-based, and safe intervention aimed at improving the overall quality of life for both male and female patients, while simultaneously reducing the long-term health risks commonly associated with the advancement of knee osteoarthritis.

### DETAILED DESCRIPTION

### Definitions

Osteoarthritis (OA) is a common joint disease, primarily affecting middle-aged to elderly individuals, involving not only cartilage but the entire joint structure, including ligaments, bone, and joint lining. It is the most prevalent form of arthritis and a leading cause of chronic pain and long-term disability in adults. Although commonly associated with older age, OA can manifest in adults under 50, underscoring the importance of early prevention to mitigate long-term quality of life reductions. The hip, knee, and hand are most frequently affected, though any joint can be involved (Loeser et al., 2012). The 2015 WHO Global Ageing and Health Report identifies osteoarthritis (OA) as a leading cause of disability in adults aged 60 and older, highlighting its significant impact on mobility and quality of life. WHO's focus on healthy aging from 2021-30 emphasizes addressing the chronic burden of OA. OA affects over 40 million individuals in the U.S. and is the nation's leading cause of disability, resulting in both direct (e.g., clinical visits, surgeries) and indirect costs (e.g., work loss) (Taruc-Uy & Lynch, 2013).

In 2020, osteoarthritis (OA) prevalence increased with age, affecting 38% of individuals aged 70 and older, with knee OA being the most common globally. The knee, the largest synovial joint, consists of bones, cartilage, ligaments, and a synovial membrane that produces fluid to nourish the cartilage. By 2050, knee OA cases are projected to rise by 74.9%, highlighting a growing global burden, with OA prevalence expected to reach 14.8% of people over 30 (Steinmetz et al., 2023). OA is classified into two groups according to its etiology: primary (idiopathic or non-traumatic) and secondary (usually due to trauma or mechanical misalignment) (Mora et al., 2018). The severity of the disease can also be graded according to the radiographic findings by the Kellgren-Lawrence (KL) system described in 1957.

Cartilage alone does not produce inflammation or pain in the early stages of osteoarthritis due to its lack of vasculature and innervation. Pain primarily arises from changes in non-cartilaginous structures like the joint capsule, synovium, subchondral bone, ligaments, and periarticular muscles. As the disease progresses, these components undergo changes such as bone remodeling, osteophyte formation, muscle weakening, ligament laxity, and synovial effusion (Mora et al., 2018). The etiology of osteoarthritis (OA) involves multiple factors, including genetic predisposition, joint injury, obesity, advanced age, knee malalignment, and sport practice, which increase the risk, especially in adolescent athletes. Knee trauma elevates the risk of knee OA. Nearly fourfold mechanical forces on joints, particularly linked to body body mass index (BMI), are modifiable risk factors. Female sex, obesity, lower education levels, and weak muscles are associated with symptomatic OA and disability. Activities involving frequent squatting, especially for prolonged periods, is a strong risk factor for tibiofemoral knee OA, doubling the risk for those who squat or kneel more than two hours a day (Musumeci et al., 2015). Additionally, meniscus surgery increases the risk of future knee OA by 2.6 times with patients undergoing partial meniscectomy or reconstruction surgery being significantly more likely to develop radiographic evidence of OA (Rongen et al., 2017).

The link between inflammation and OA is complex, with ongoing debate about whether inflammation triggers OA or is a secondary effect. Unlike inflammatory arthritis, OA features chronic, low-grade inflammation primarily driven by innate immune mechanisms. Synovitis, characterized by inflammatory cell infiltration in the synovium, is common in OA and increases with disease severity. Synovial fluid in OA contains inflammatory mediators like C-reactive protein, prostaglandins, and cytokines, which can stimulate matrix metalloproteinases and lead to cartilage breakdown (Sokolove & Lepus, 2013).

Chronic inflammation involving white blood cells can result in further tissue destruction. Damage to the extracellular matrix releases molecules that activate innate immune cells, with studies showing macrophages play a role in osteophyte formation, a key feature of OA. While the body has protective growth factors, these mechanisms may be altered in knee OA patients, potentially harming joint health (Robinson et al., 2016).

Pain is the most prevalent symptom and a significant cause of chronic disability associated with OA. It can range from mild to debilitating and is typically worsened by activity and alleviated by rest. In advanced OA, synovitis may develop, causing pain even at rest. Patients often experience short morning stiffness lasting less than 30 minutes and may show tenderness upon palpation of the affected joints. Joint effusions can occur, usually characterized by mild pleocytosis, normal viscosity, and modestly elevated protein levels. Limitations in the range of motion are common, and in severe cases, malalignment may be observed (e.g., genu varum or genu valgum) (Hunter et al., 2008).

OA pain mainly arises from the synovium and subchondral bone, as these areas, unlike normal articular cartilage lack nerve innervation, contain sensory nerve fibers in the meniscus, ligaments, tendons, and fat pads (Neogi, 2013). Nociceptors in these tissues respond to mechanical, chemical, or thermal stimuli, transmitting pain signals through the dorsal root ganglion and spinal cord to the brain. Additionally, the persistent inflammatory environment in OA leads to peripheral and central nerve sensitization, characterized by mechanical allodynia and hyperalgesia around the affected joint (Tong et al., 2022).

Some of the pharmacological interventions for knee OA include oral Nonsteroidal Anti-inflammatory Drugs (NSAIDs), that inhibit cyclooxygenase (COX) enzymes, which reduce proinflammatory eicosanoids like prostaglandins. NSAIDs come in two forms: nonselective, affecting both COX-1 (which maintains gastrointestinal and kidney function) and COX-2 (involved in inflammation), and COX-2 selective drugs like celecoxib, which aim to reduce inflammation with fewer gastrointestinal side effects. While effective, NSAIDs pose risks such as gastrointestinal ulcers, renal dysfunction, and cardiovascular issues, including myocardial infarction and stroke, with diclofenac linked to higher cardiovascular risks. Other potential side effects include liver toxicity, bleeding complications, and skin or respiratory reactions, particularly in individuals with aspirin-exacerbated respiratory disease (Zarghi & Arfaei, 2011).

Moreover, there are five FDA-approved injectable corticosteroids for intra-articular (IA) injections: methylprednisolone acetate, triamcinolone acetate, triamcinolone hexacetonide, betamethasone (acetate and sodium phosphate) and dexamethasone. These corticosteroids exert anti-inflammatory and immunosuppressive effects by acting on nuclear steroid receptors, leading to reduced inflammation, vascular permeability, and the synthesis of pain mediators. Although they are effective for symptomatic OA pain, their benefits are typically short-lived, averaging 2-4 weeks, prompting up to four injections per year (McAlindon et al., 2017).

IA injections have been linked to local and systemic side effects. Local immediate side effects include postinjection flare, which is an increase in pain after the injection, occurring in 32.8-35.3% of patients, particularly with particulate steroids Local delayed side effects can involve skin hypopigmentation and atrophy, affecting around 10% of patients, although this typically resolves within a year (Fawi et al., 2017). Soft-tissue calcification may also occur, resulting in increased pain scores (Park et al., 2019). Additionally, accelerated osteoarthritis progression is noted, with increased joint space loss, particularly in the knees (Zeng et al., 2019). Osseous injury can occur due to osteocyte apoptosis and related conditions like osteonecrosis (Graf et al., 2022).

Systemic delayed side effects can involve adrenal insufficiency, which can be life-threatening and may be present in 25.3% of patients 6 months after injection, alongside osteoporosis linked to long-term corticosteroid use and resulting in dose-dependent effects on bone density (Kim et al., 2016).

Non-pharmacological interventions for knee osteoarthritis include canes and braces for pain relief and support, supervised exercise to improve pain and function, and weight loss to reduce pain in obese patients. Massage therapy and acupuncture provide additional pain relief, while electrical therapies like TENS and PENS modulate pain through nerve stimulation. Extracorporeal Shockwave Therapy (ESWT) aids tissue regeneration and reduces inflammation (American Academy of Orthopaedic Surgeons, 2021).

The OA Research Society International (OARSI) recommends conservative treatment as the first step for knee osteoarthritis (OA), with surgery considered only if symptoms persist after nonsurgical interventions. Surgical options include arthroscopic debridement, cartilage repair surgery, osteotomy, and unicompartmental or total knee arthroplasty, depending on symptoms, OA stage, and patient factors like age and comorbidities. Arthroscopy is widely used, though its efficacy compared to other treatments is debated, with some studies showing limited benefits. Patient selection is crucial, as better outcomes are observed in those with less extensive arthritis. While arthroscopic lavage may provide short-term relief for select patients with mild radiographic OA or symptomatic meniscal tears, it should not be routinely performed for knee OA due to a lack of evidence for significant benefits. Overall, the procedure can be performed as an outpatient with low complication risk, but it does not alter OA progression.

Endocannabinoid system: The endocannabinoid system (ECS) plays an important role in the regulation of homeostatic balance, which is responsible for maintaining the body's physiological functions. This system is composed of cannabinoid receptors, endocannabinoids, and enzymes that interact with each other, causing reactions in the cells, that is to say when some alteration occurs, the human body starts to synthesize endocannabinoids that usually cooperate with the cannabinoid receptors, this activation produces changes inside the cells that originate stability in the vital and physiological processes (Di Marzo 2004).

CB1, and CB2 cannabinoid receptors are the best characterized. They differ in the way they transmit the signal and in their distribution in the different tissues. Both are G protein-coupled receptors (GPCRs) and their activation leads to the inhibition of adenylyl cyclase, which prevents the conversion of ATP to cyclic AMP (cAMP) and prevents certain voltage-sensitive calcium channels, as well as stimulating mitogen-activated protein kinases (MAP kinases) and inwardly rectifying potassium channels (GIRKs), and recruiting beta-arrestins, among other actions (Howlett & Abood, 2017).

These receptors differ in signal transmission and tissue distribution. CB1, mainly in the brain and nervous system, inhibits neurotransmitter release, while CB2, found in immune cells, modulates inflammation. Other related receptors include TRPV and GPR types. Endocannabinoids are long-chain fatty acid neurotransmitters like anandamide (AEA) and 2-AG, synthesized and released as needed.

Cannabinoid receptors and joint health: Bone remodeling involves osteoblasts and osteoclasts; metabolic imbalances can lead to osteoporosis. Cannabinoids regulate bone metabolism via CB1R and CB2R. Activation of CB2R can reduce bone loss without significant psychoactive effects. CB2R influences various cellular processes, such as regulating calcium levels and inhibiting NF-κB, affecting bone resorption and remodeling. Research shows that CB2R signaling is crucial for maintaining bone mass and may be a target for osteoporosis treatment (Geng et al., 2023)

Cannabichromene (CBC): CBC is one of three major cannabinoids enzymatically produced from CBG by Cannabis plants, along with THC and CBD. It acts as a CB2 agonist and has low potency at TRPM8 channels. It demonstrates significant antinociceptive effects in various pain models (Raup-Konsavage et al., 2023), without inducing catalepsy like THC. Additionally, CBC binds to CB1 receptors and peroxisome proliferator-activated receptors (PPARs) (Granja, A. G. 2012).

Studies using artificial intelligence (AI) and animal models have demonstrated CBC's effectiveness in reducing various types of pain, including neuropathic, acute, inflammatory, and heat-induced pain (Raup-Konsavage et al., 2024). Unlike THC, CBC does not induce catalepsy, making it a more suitable candidate for pain management without the psychoactive effects associated with high levels of THC.

CBC also functions as an inhibitor of the MAPK and NF-κB pathways to reduce inflammatory markers and nitric oxide production, suggesting that this phytocannabinoid could be a valuable treatment for conditions involving chronic or acute inflammation (Hong et al., 2023) such as osteoarthritis.

Cannabigerol (CBG): CBG is a cannabinoid from the Cannabis sativa plant that lacks psychotomimetic effects and serves as a precursor to CBC, CBD and THC. Potential pharmacological targets of CBG include transient receptor potential (TRP) channels, cyclooxygenase enzymes (COX-1 and COX-2) and cannabinoid, 5-HT1A and alpha-2 receptors. Preclinical findings show that CBG reduces intraocular pressure, possesses antioxidant, anti-inflammatory, and antitumor activities, and has anxiolytic, neuroprotective, dermatological, and appetite-stimulating effects (Calapai F 2022).

CBG reduces inflammation by decreasing myeloperoxidase activity and inducible nitric oxide synthase (iNOS) expression, while also modulating cytokine levels, lowering pro-inflammatory markers like IL-1β and IFN-γ, and increasing the anti-inflammatory cytokine IL-10. CBG's anti-inflammatory effects are mediated primarily through CB2 receptor activation, as blocking CB2R diminishes these effects (Borrelli, I. 2013). Additionally, CBG enhances antioxidant defenses by increasing superoxide dismutase (SOD) activity, reducing reactive oxygen species (ROS), and downregulating iNOS and nitrotyrosine. It has demonstrated comparable antioxidant activity to vitamin E and protects cells from oxidative stress, including hydrogen peroxide-induced damage. Furthermore, CBG shows neuroprotective potential by reducing apoptosis, increasing anti-apoptotic protein Bcl-2, and protecting neurons from oxidative and inflammatory stress. These combined anti-inflammatory, antioxidant, and neuroprotective effects suggest that CBG could be valuable in treating disorders related to inflammation, oxidative stress, and neurodegeneration (Gugliandolo, F. 2018).

Hyaluronic Acid (HA): HA is a natural glycosaminoglycan that improves the elasticity and moisture of cartilage, inhibits chondrocyte apoptosis, and stimulates proteoglycan synthesis, playing a crucial role in preventing cartilage erosion and reducing synovial inflammation. In osteoarthritis (OA) patients, the depolymerization and clearance rates of endogenous HA are higher, decreasing its concentration and viscosity in synovial fluid. The use of exogenous HA through intra-articular injections helps restore these properties and is recommended by OARSI for knee OA treatment. Research has shown that HA can be an effective option compared to NSAIDs, corticosteroid injections, and PRP, with persistent improvements in knee pain and function (Moreland, 2003).

Hyaluronic acid is an important modulator, especially through interaction with CD44 receptors that are present in fibroblast-like synoviocytes. Therefore, in addition to the mechanical effects of promoting better force distribution, diminishing the pressure due to weight and recovering the rheological properties of the synovial fluid (Bagga, H. 2006), hyaluronic acid also acts biochemically. It diminishes the gene expression of the cytokines and enzymes that are associated with osteoarthritis, and diminishes prostaglandin production and the intra-articular metalloproteinase concentration.

Hydrolyzed Collagen: Collagen is a major structural protein in the human body, representing the main component of connective tissue and constituting approximately 45-75% of dry weight in the ligaments, tendons, and cartilage. Exposure to collagen preparations can induce chondrocyte proliferation and increase HA production while reducing inflammatory mediators.

Intra-articular collagen injections have been shown to delay cartilage degeneration in osteoarthritis (OA) by promoting cartilage repair and reducing inflammation (Naraoka et al., 2013). OA joints experience structural damage and inflammation, which activates enzymes like matrix metalloproteinases (MMPs) and ADAMTS that degrade the extracellular matrix (ECM), including collagen. Exogenous collagen injections can help counteract this degradation by stimulating the production of hyaluronic acid and decreasing inflammation mediators such as IL-1β and TNF-β (Loeser et al., 2012).

Calcium gluconate: Calcium gluconate is a water-soluble calcium salt that has been researched for its anti-inflammatory, antioxidant, and immunomodulatory properties. Specifically, calcium gluconate has demonstrated the ability to reduce pro-inflammatory cytokines such as IL-6 and TNF-α, suggesting its capacity to modulate the immune response and alleviate inflammation. Moreover, its antioxidant properties are crucial for managing oxidative stress, a key factor in the pathogenesis of various inflammatory conditions. By regulating the production of cytokines involved in the immune response, it leads to a balanced immune reaction that prevents excessive inflammation and promotes tissue repair. Applications of calcium gluconate in medicine range from the treatment of inflammatory diseases like rheumatoid arthritis and osteoarthritis, to protection against bone loss, especially during aging (Sohn et al., 2013).

### Pharmaceutical Compositions

Aspects of the disclosure provide a method to treat symptoms associated with osteoarthritis and maintain optimal balance health in patient using a nanoplatform composition using nanoemulsion of phytocannabinoids (CBC, CBG), hyaluronic acid or related compounds thereof, hydrolyzed collagen or related compounds thereof and calcium gluconate supplement or related compounds thereof specifically aimed at patients who have knee osteoarthritis.

The disclosure, as described in the examples provided, is not limited to the details shown as modifications can be made without departing from the scope of the disclosure and the equivalent of the claims. The following description of specific embodiments will help clarify the construction, operation, and additional benefits of the disclosure.

Aspects of the disclosure refer to a composition for the treatment of osteoarthritis, comprising: a) an active principle comprising at least two cannabinoids; b) hyaluronic acid or related compounds thereof, hydrolyzed collagen or related compounds thereof, calcium gluconate or related compounds thereof, or combinations thereof and c) a stabilizing solubilizing agent.

In some embodiments, the active principle comprises at least one cannabinoid selected from a range of cannabinoids including Δ9-Tetrahydrocannabinol (THC), Cannabidiol (CBD), Cannabigerol (CBG), Cannabinol (CBN), Tetrahydrocannabivarin (THCV), Cannabichromene (CBC), Delta-8-Tetrahydrocannabinol (Δ8-THC), Cannabidivarin (CBDV), Tetrahydrocannabinolic Acid (THCA), Cannabidiolic Acid (CBDA), Cannabigerolic Acid (CBGA), and Cannabichromenic Acid (CBCA). These cannabinoids may be used individually or in specific combinations to achieve targeted therapeutic effects, tailored to the needs of the patient. For instance, the selection of cannabinoids like CBD and CBG may be prioritized for their strong anti-inflammatory and neuroprotective properties, making them highly suitable for managing the symptoms of knee osteoarthritis, such as inflammation and cartilage degradation.

The incorporation of multiple cannabinoids can be further optimized to leverage the entourage effect, wherein the combination of cannabinoids enhances their individual therapeutic properties. This synergistic interaction can amplify their efficacy in reducing inflammation, modulating immune responses, and providing chondroprotective benefits, which are essential for preventing cartilage breakdown and promoting regeneration. In particular, cannabinoids such as CBC and CBG can be combined with CBD or THCV to provide a broad-spectrum anti-inflammatory response, which is critical in mitigating the joint pain and stiffness associated with osteoarthritis.

In certain embodiments, the cannabinoid composition can be specifically tailored to focus on key physiological pathways affected by osteoarthritis, such as pain management, inflammation control, and joint lubrication. For example, combining CBD with CBGA or CBN may enhance pain relief while offering additional benefits like reducing oxidative stress and promoting cartilage regeneration. Meanwhile, incorporating cannabinoids like THC or THCV may provide more potent analgesic effects, particularly for patients with severe pain that does not respond adequately to traditional therapies.

Moreover, the formulation may be designed to vary the ratios of these cannabinoids depending on the stage of the disease and the individual's response to treatment. For instance, higher concentrations of CBD and CBG may be favored in the early stages of osteoarthritis to reduce inflammation and prevent further joint degeneration, while cannabinoids like CBC and THCA may be added in later stages to enhance chondroprotection and support tissue repair. This flexibility in the cannabinoid composition allows for a more personalized treatment approach, adapting to the progression of the disease and the unique therapeutic needs of the patient.

In addition, the nanoplatform delivery system ensures that these cannabinoids are delivered efficiently, with improved bioavailability and controlled release. This allows the active ingredients to remain in the affected joint for an extended period, providing sustained relief and promoting long-term cartilage health. By tailoring the cannabinoid composition to specific therapeutic goals and utilizing the nanoplatform for enhanced delivery, the formulation offers a comprehensive solution for managing the symptoms and progression of knee osteoarthritis and other related conditions.

In various embodiments, the composition may vary in the concentration of cannabinoids to tailor therapeutic potency and patient response, allowing for precise adjustment based on the condition being treated, patient tolerance, and desired therapeutic outcomes. For instance, higher concentrations of cannabinoids may be employed for individuals experiencing severe pain and inflammation associated with knee osteoarthritis, whereas lower concentrations might be suitable for patients with milder symptoms or those who are sensitive to cannabinoid effects.

Additionally, the ratio of different cannabinoids within the composition can be adjusted to optimize the therapeutic profile while maintaining efficacy. This customizable approach enables specific formulation strategies, such as emphasizing the therapeutic effects of certain cannabinoids to target particular symptoms or physiological pathways. For example, increasing the proportion of cannabigerol (CBG) relative to cannabichromene (CBC) may enhance anti-inflammatory and neuroprotective effects, while a higher ratio of CBC could amplify analgesic properties to better manage pain.

In further embodiments, the composition may combine two or more cannabinoids in specific ratios to produce a synergistic effect, where the combined therapeutic action is greater than the effect of individual cannabinoids alone. This phenomenon, often referred to as the “entourage effect,” leverages the complementary mechanisms of different cannabinoids to enhance overall efficacy. By modulating various physiological pathways simultaneously, the synergistic combination can address the complex pathology of osteoarthritis more effectively. For example, a composition with a high CBC-to-CBG ratio might be formulated to maximize anti-inflammatory and analgesic effects, providing improved mobility and relief from both local and general discomfort. CBC is known for its ability to interact with the endocannabinoid system and reduce inflammation by inhibiting pro-inflammatory cytokines. When combined with CBG, which has demonstrated neuroprotective and anti-inflammatory properties, the two cannabinoids can work together to more effectively reduce joint inflammation and pain.

Alternatively, a composition with a balanced ratio of CBD, CBG, and CBC could be designed to target multiple symptoms simultaneously. CBD (Cannabidiol) adds another layer of therapeutic potential due to its well-documented anti-inflammatory, analgesic, and anxiolytic effects. The inclusion of CBD could enhance the overall anti-inflammatory response, reduce anxiety associated with chronic pain, and improve sleep quality, which is often disrupted in patients with osteoarthritis. Moreover, the composition might include minor cannabinoids like CBN (Cannabinol) or THCV (Tetrahydrocannabivarin) in small but significant amounts to further enhance the therapeutic profile. CBN has sedative properties that could aid in sleep, while THCV has been studied for its potential anti-inflammatory and bone-stimulating effects, which could contribute to bone health and cartilage preservation.

By carefully adjusting the concentrations and ratios of cannabinoids, the composition can be tailored to: Enhance Anti-inflammatory Effects: Higher concentrations of CBG and CBD can synergistically inhibit inflammatory mediators like TNF-α, IL-1β, and IL-6, reducing joint inflammation and slowing cartilage degradation. Improve Pain Management: Increasing the proportion of CBC and CBD may amplify analgesic effects by modulating pain perception pathways, providing more effective relief from chronic joint pain. Promote Cartilage Repair and Bone Health: Incorporating cannabinoids like CBGA (Cannabigerolic Acid), alongside CBG, may stimulate chondrocyte activity and support cartilage regeneration, while cannabinoids influencing osteoblast and osteoclast function can help maintain bone density. Address Neuropathic Pain: Including cannabinoids that target TRPV1 receptors, such as CBG, can alleviate neuropathic pain often associated with osteoarthritis by desensitizing pain receptors.

This strategic combination and customization enable the formulation to address the multifaceted nature of osteoarthritis more comprehensively. By targeting inflammation, pain, cartilage degradation, and bone density loss concurrently, the composition offers a holistic approach to treatment. Furthermore, the use of a nanoplatform delivery system enhances the bioavailability of the cannabinoids, ensuring that the adjusted concentrations and ratios are effectively delivered to the target tissues within the joint. The nanoplatform facilitates controlled release, maintaining therapeutic levels of cannabinoids over an extended period, which is particularly beneficial for chronic conditions like osteoarthritis. In conclusion, varying the concentration and ratios of cannabinoids in the composition allows for a flexible and personalized treatment strategy. This approach maximizes therapeutic benefits through synergistic effects while minimizing potential side effects, ultimately improving patient outcomes and quality of life for those suffering from knee osteoarthritis.

In various embodiments, the composition may vary in the concentration of cannabinoids to tailor therapeutic potency and patient response, allowing for precise adjustment based on the condition being treated, patient tolerance, and desired therapeutic outcome. For example, higher concentrations of cannabichromene (CBC) may be employed for its anti-inflammatory and anxiolytic properties, whereas compositions with increased levels of tetrahydrocannabinol (THC) might be suited for enhanced analgesic effects in patients managing chronic pain. Additionally, varying concentrations of cannabidiol (CBD) may be included for its neuroprotective and anti-seizure properties, providing a synergistic effect when combined with CBC for broader anti-inflammatory benefits.

The composition can also be adjusted to incorporate cannabigerol (CBG), particularly in patients who require neuroprotective effects or who are suffering from bacterial-induced inflammation, as CBG has demonstrated antimicrobial properties. CBG's inclusion can also be beneficial for promoting cartilage regeneration, which is essential for patients experiencing joint degradation in osteoarthritis.

Further, the ratio of cannabinoids can be customized for targeted conditions, such as focusing on a higher ratio of CBC to CBG for enhanced anti-inflammatory action, or incorporating a balanced blend of THC and CBD for a combination of pain relief and anti-inflammatory effects with reduced psychoactive impact. Such flexibility in cannabinoid concentration and ratio allows for personalized treatments tailored to the specific needs and responses of individual patients, ensuring maximum therapeutic efficacy while minimizing side effects. In more advanced cases of osteoarthritis, higher concentrations of both CBC and CBG may be necessary to effectively manage chronic inflammation and prevent further joint damage.

Additionally, the ratio of different cannabinoids within the composition can be adjusted to create personalized treatment profiles, allowing for a customizable approach based on the specific therapeutic needs of each patient. This customizable approach enables specific formulation strategies, such as emphasizing the therapeutic effects of CBC (cannabichromene) and CBG (cannabigerol) for targeted outcomes, such as enhanced anti-inflammatory activity or neuroprotection.

For instance, a higher concentration of CBC may be favored in formulations aimed at reducing chronic inflammation associated with osteoarthritis, as CBC is known for its potent anti-inflammatory and analgesic properties, which can also help reduce symptoms like joint stiffness and swelling. Additionally, CBC has been shown to interact with TRPV1 and TRPA1 receptors, which are involved in nociception, further supporting its role in pain relief. On the other hand, CBG is particularly valuable for its neuroprotective properties, which can be essential for patients suffering from conditions that involve nerve damage or degeneration. CBG has also demonstrated antimicrobial and antioxidant effects, making it a key component in promoting cartilage regeneration and slowing down disease progression in osteoarthritis.

The composition includes cannabinoids at concentrations ranging from 0.5% to 5%, with a preferred embodiment containing cannabinoids at concentrations between 0.65% and 3%, and more preferably the composition includes cannabinoids at concentrations ranging from 2% to 3%, ensuring precision in therapeutic dosing. This range allows for flexible adjustments depending on the severity of the condition being treated. For example, in early-stage osteoarthritis or mild inflammatory conditions, lower concentrations of cannabinoids might suffice. However, in more advanced cases, higher concentrations within the preferred range may be necessary to provide sufficient anti-inflammatory, analgesic, and regenerative effects.

In addition to CBC and CBG, the ratio of other cannabinoids such as cannabidiol (CBD) or tetrahydrocannabinol (THC) can also be adjusted within the composition to achieve specific therapeutic goals. For example, a composition with a higher ratio of CBD might be preferred for its anti-anxiety, anti-inflammatory, and neuroprotective properties, while reducing the psychoactive effects that are commonly associated with THC. Meanwhile, THC, when present in carefully controlled concentrations, can contribute to the overall analgesic effect of the composition, particularly in patients suffering from more severe pain.

Furthermore, the “entourage effect,” where cannabinoids work synergistically to enhance each other's therapeutic effects, is a critical consideration when adjusting the cannabinoid ratio. This effect allows the formulation to provide a broader therapeutic spectrum, addressing not only pain and inflammation but also underlying issues such as cartilage degradation and bone loss in osteoarthritis patients.

By tailoring the cannabinoid ratios within this range, the composition can be optimized for various routes of administration, such as intra-articular injections, ensuring that the active ingredients are delivered effectively to the target tissues while minimizing systemic side effects. The flexibility in cannabinoid concentrations and ratios allows healthcare providers to customize treatments that can be adjusted over time, depending on patient response and evolving clinical needs, ensuring that the therapeutic effects are sustained throughout the treatment period.

The composition may combine two or more cannabinoids in specific ratios that yield a synergistic effect, where the therapeutic action of the combination exceeds the benefits of each cannabinoid individually. This synergistic effect, often referred to as the “entourage effect,” enables the composition to address complex conditions more effectively by modulating various physiological pathways simultaneously.

The entourage effect arises from the interaction between different cannabinoids and the body's endocannabinoid system (ECS), which regulates a wide range of physiological functions such as pain perception, inflammation, and immune response. When cannabinoids like CBC (cannabichromene) and CBG (cannabigerol) are combined, they can amplify each other's therapeutic effects through their interaction with specific cannabinoid receptors, primarily CB1 and CB2, as well as other receptors like TRPV1 and TRPA1. This combined action not only enhances pain relief but also targets inflammation, tissue regeneration, and neuroprotection, making it a highly effective approach in treating conditions like osteoarthritis.

For instance, combining CBC and CBG in a particular ratio could potentiate pain relief while simultaneously reducing inflammation and promoting joint health. CBC, with its known anti-inflammatory and analgesic properties, works by inhibiting the production of pro-inflammatory cytokines and interacting with TRPV1 receptors, which play a role in pain signaling. CBG, on the other hand, has shown neuroprotective effects and the ability to stimulate the production of natural endocannabinoids, which further reduce inflammation and support cartilage regeneration. Together, these cannabinoids can provide not only effective pain control but also improved mobility and relief of both local and general discomfort in patients suffering from degenerative joint conditions.

In further embodiments, the composition may combine two or more cannabinoids in specific ratios tailored for particular therapeutic goals. For example, a high CBC-to-CBG ratio may be employed to prioritize anti-inflammatory effects and pain control while also supporting cartilage regeneration and overall joint function. This tailored approach allows the composition to be fine-tuned to address the specific needs of patients with varying levels of osteoarthritis progression or inflammation severity. By adjusting the ratio of CBC to CBG, the composition can either focus on reducing acute inflammation and pain or provide long-term support for joint health and mobility, depending on the patient's condition and treatment objectives.

These tailored ratios are designed to produce a synergistic effect, where the combined therapeutic action of cannabinoids is greater than the effect of each individual cannabinoid alone. This approach not only enhances pain relief and reduces inflammation but also supports overall joint health by promoting the regeneration of cartilage and reducing the risk of further joint degradation. The use of multiple cannabinoids in specific ratios can also help minimize the risk of side effects commonly associated with higher doses of individual cannabinoids, such as the psychoactive effects of THC, making the treatment more tolerable for a wider range of patients.

In some embodiments, the included cannabinoids are CBC (cannabichromene), CBG (cannabigerol), or combinations thereof. These cannabinoids are carefully selected for their individual and synergistic properties, with CBC known for its strong anti-inflammatory and analgesic effects, and CBG offering neuroprotective and cartilage-regenerating properties. This combination can be especially effective in treating osteoarthritis, where both pain relief and joint preservation are critical to improving the patient's quality of life.

The composition may also be formulated for intra-articular injection, with cannabinoids included at specific concentrations to optimize therapeutic efficacy. In some embodiments, the composition includes cannabinoids at a concentration ranging from 0.83% to 0.92% of CBC and 1.50% to 1.65% for CBG, ensuring precision in dosing for each patient. These concentrations are designed to maximize the therapeutic benefits while minimizing the risk of side effects, making it suitable for regular use in managing chronic conditions like osteoarthritis.

The intra-articular route of administration ensures that the cannabinoids are delivered directly to the affected joint, where they can exert their effects most efficiently. This targeted approach allows for lower systemic exposure to cannabinoids, reducing the likelihood of unwanted side effects while providing concentrated relief at the site of inflammation and joint degeneration. By delivering the cannabinoids directly into the synovial fluid, the composition can help improve joint lubrication, reduce pain and stiffness, and promote long-term joint health through the regeneration of cartilage and reduction of inflammation. The combination of CBC and CBG in specific ratios within the composition allows for a highly flexible and effective treatment option. The synergy between these cannabinoids enhances their individual therapeutic properties, providing comprehensive relief for pain, inflammation, and joint degradation. Whether administered through intra-articular injection or other routes, the composition is designed to offer long-term support for joint health and mobility, making it an ideal solution for patients suffering from osteoarthritis and related conditions.

In some embodiments, the composition is formulated with specific concentrations of cannabinoids, carefully balanced to optimize therapeutic outcomes. In some embodiments, the included cannabinoids are cannabichromene (CBC), cannabigerol (CBG) or combinations thereof. These cannabinoids may be selected for their individual or synergistic therapeutic properties, with CBC known for its anti-inflammatory and analgesic effects, and CBG for its neuroprotective, anti-bacterial and cartilage-regenerating properties.

In one non-limiting embodiment, this composition for osteoarthritis care contains the following active ingredients per dosage: CBC—25 mg; CBG—45 mg. However, other embodiments of the disclosure include variations in the formulation to allow for more precise tailoring based on patient needs, therapeutic goals, and tolerability, optimizing the treatment's effectiveness while minimizing potential side effects.

In another embodiment of the disclosure, the composition could contain CBC: 22.5 mg and CBG: 40.5 mg. This lower-range variation may be suitable for patients who are more sensitive to cannabinoids or are in the early stages of osteoarthritis, where a lower potency is sufficient. By reducing the cannabinoid concentration, this formulation would provide therapeutic benefits while minimizing risks of adverse effects like drowsiness or cognitive impairment.

In yet another embodiment, the composition might include CBC: 23.75 mg and CBG: 42.75 mg. This slight reduction in concentration could benefit patients who respond well to cannabinoids but experience mild side effects at higher doses. This allows for effective symptom management while reducing the cannabinoid load slightly for long-term treatment tolerability.

A further embodiment of the disclosure keeps the standard dosage of CBC: 25 mg and CBG: 45 mg. This is the recommended composition for managing moderate to severe osteoarthritis, providing strong anti-inflammatory and analgesic effects suited for patients with significant joint pain, stiffness, and reduced mobility due to advanced osteoarthritis.

Another embodiment could include CBC: 26.25 mg and CBG: 47.25 mg. This increase in concentration could be helpful for patients requiring stronger symptom management, such as those whose condition has progressed beyond the relief provided by the standard dosage, yet without moving to the highest concentration.

In yet another embodiment of the disclosure, the composition might contain CBC: 27.5 mg and CBG: 49.5 mg. This higher concentration is intended for patients experiencing severe symptoms of osteoarthritis, including chronic inflammation, extreme pain, and significant loss of mobility. The increased levels of CBC and CBG offer enhanced anti-inflammatory and pain-relieving effects.

Finally, various combinations within these ranges could be customized according to individual patient needs. For example, a higher dose of CBC (27.5 mg) combined with a moderate dose of CBG (42.75 mg) could prioritize reducing inflammation, while a higher dose of CBG (49.5 mg) combined with a lower dose of CBC (23.75 mg) might be ideal for managing intense pain with moderate inflammation. This tailored approach ensures that the formulation provides optimal relief and joint support, depending on the specific symptoms and disease progression of the patient.

These various embodiments allow the treatment to be adapted to the evolving needs of patients, ensuring that cannabinoids are administered at the most effective and safest concentrations for long-term management of osteoarthritis symptoms.

In one preferred embodiment, the composition comprises the following concentrations: CBC at 0.83%, and CBG at 1.5%. However, other embodiments of the disclosure explore variations in cannabinoid concentrations to better tailor therapeutic effects based on patient needs, treatment goals, and response to therapy, ensuring a flexible and effective approach to managing osteoarthritis symptoms.

In one embodiment of the disclosure, the composition could include a lower concentration of CBC at 0.5% and CBG at 1%. This formulation might be suitable for patients in the early stages of osteoarthritis or those who are particularly sensitive to cannabinoids, allowing for effective symptom relief while minimizing the risk of side effects, such as drowsiness or cognitive impairment.

Another embodiment might involve CBC at 0.75% and CBG at 1.25%, offering a slight increase in cannabinoid content while remaining on the lower end of the concentration spectrum. This variation could be ideal for patients who need more robust symptom management but still benefit from a conservative dosage to avoid potential tolerance buildup or adverse effects.

A further embodiment maintains the preferred concentrations of CBC at 0.83% and CBG at 1.5%, providing a balanced formulation suitable for managing moderate osteoarthritis symptoms. This combination supports effective pain relief and anti-inflammatory action, particularly for patients experiencing persistent knee pain, stiffness, and mobility issues.

In yet another embodiment, the composition could contain CBC at 1.2% and CBG at 2%. This higher concentration might be beneficial for patients with more advanced osteoarthritis, where greater cannabinoid potency is necessary to manage more severe pain and inflammation. This formulation provides stronger therapeutic effects without exceeding tolerability limits.

In another embodiment, the composition could include CBC at 1.5% and CBG at 2.25%, offering enhanced pain-relieving and anti-inflammatory properties. This ratio would be advantageous for patients whose osteoarthritis has progressed significantly, requiring a more aggressive approach to symptom management.

Finally, in the upper range of cannabinoid concentrations, the composition could contain CBC at 2% and CBG at 2.5%. This high-concentration formulation would be particularly effective for patients suffering from severe osteoarthritis, where both pain and inflammation are chronic and debilitating. The increased cannabinoid levels provide maximum therapeutic benefit, helping to alleviate pain, improve joint function, and slow disease progression.

These different embodiments, ranging from lower to higher concentrations of CBC and CBG, allow for precise and personalized treatment strategies. The composition can be adjusted to the patient's evolving needs, ensuring that cannabinoids are delivered in concentrations that offer optimal relief while maintaining safety and tolerability across various stages of osteoarthritis.

In further embodiments, the ratio of cannabinoids can be adjusted within these concentration ranges to tailor the composition to specific patient needs or therapeutic goals, providing flexibility in managing a wide spectrum of osteoarthritis symptoms. In another embodiment, the composition may be combined with major components of the extracellular matrix in connective tissues like structural proteins such as hydrolyzed collagen, glycosaminoglycans such as hyaluronic acid and mineral supplement such as calcium gluconate.

In further embodiments, the ratio of cannabinoids can be adjusted within these concentration ranges to tailor the composition to specific patient needs or therapeutic goals, providing flexibility in managing a wide spectrum of osteoarthritis symptoms. This customizable approach allows the composition to address the varying severities and manifestations of osteoarthritis. For instance, some patients may require a formulation with a higher concentration of CBC (cannabichromene) for its anti-inflammatory properties, particularly in cases where inflammation is the dominant symptom. Others may benefit from a higher CBG (cannabigerol) content, which is known for its neuroprotective and analgesic effects, especially for those experiencing significant nerve-related pain or cartilage degeneration. The ability to adjust the ratio of cannabinoids ensures that the therapeutic approach can be fine-tuned to deliver maximum effectiveness for individual cases.

This flexibility is particularly beneficial when managing the progressive nature of osteoarthritis. As the condition evolves, the therapeutic needs of patients may shift. Initially, a higher dose of CBC might be necessary to control inflammation, while later in the disease progression, a greater concentration of CBG could be prioritized to manage pain and improve mobility. The ability to adjust cannabinoid ratios ensures that the treatment can be adapted over time, allowing for sustained efficacy as the condition changes.

In another embodiment, the composition may be combined with major components of the extracellular matrix in connective tissues, such as structural proteins like hydrolyzed collagen, glycosaminoglycans like hyaluronic acid, and mineral supplements such as calcium gluconate. These components are crucial for providing comprehensive joint support, addressing not only the symptoms of osteoarthritis but also the structural damage caused by the disease.

Hydrolyzed collagen is a key structural protein that plays an essential role in maintaining and repairing cartilage, tendons, and ligaments. In patients with osteoarthritis, the breakdown of cartilage leads to joint pain, stiffness, and loss of function. By including hydrolyzed collagen in the composition, the formulation supports the regeneration of cartilage tissue, promoting chondrocyte proliferation and the synthesis of new extracellular matrix components. This helps to restore the structural integrity of the joint, slow the progression of cartilage degradation, and improve overall joint function. The inclusion of hydrolyzed collagen is especially beneficial for patients with advanced osteoarthritis, where cartilage loss is significant.

Hyaluronic acid, a naturally occurring glycosaminoglycan in the body's synovial fluid, plays a critical role in lubricating joints and maintaining smooth movement. In osteoarthritis, the natural levels of hyaluronic acid in the joints decrease, resulting in increased friction, pain, and further degradation of the cartilage. Incorporating exogenous hyaluronic acid into the composition helps to replenish the synovial fluid, reduce friction within the joint, and improve lubrication. This not only alleviates pain and stiffness but also protects the remaining cartilage from further wear and tear. Additionally, hyaluronic acid has been shown to have anti-inflammatory properties, making it an ideal complement to the cannabinoid components of the composition, enhancing the overall therapeutic effects by reducing inflammation while promoting joint lubrication and flexibility.

In further embodiments, the composition can also include mineral supplements such as calcium gluconate. Osteoarthritis not only affects cartilage but also the subchondral bone, leading to a decrease in bone density and an increased risk of fractures. Calcium gluconate supports bone health by helping to maintain bone mineral density, strengthen the bone matrix, and reduce the likelihood of osteoporotic fractures, which are common in patients with long-term osteoarthritis. This mineral supplement provides essential support for the structural integrity of bones, working synergistically with the cannabinoids, collagen, and hyaluronic acid to offer a more comprehensive solution for managing the degenerative effects of osteoarthritis.

The combination of cannabinoids with these structural and mineral components addresses both the symptomatic relief and the underlying causes of osteoarthritis. The cannabinoids help to control pain and inflammation, while the hydrolyzed collagen and hyaluronic acid work to restore cartilage and improve joint function. Calcium gluconate, in turn, supports bone health, ensuring that the entire joint structure is protected and strengthened. This multi-modal approach not only improves quality of life by reducing pain and enhancing mobility but also offers long-term benefits by slowing disease progression and promoting tissue regeneration.

By adjusting the cannabinoid ratios and incorporating structural proteins, glycosaminoglycans, and mineral supplements, this composition provides a comprehensive therapeutic strategy for osteoarthritis patients. It addresses the complex pathophysiology of the disease, offering both immediate symptom relief and support for long-term joint and bone health. This tailored approach ensures that the treatment can be adapted to meet the unique needs of each patient, delivering a personalized and effective solution for managing osteoarthritis across its various stages.

When developing therapeutic formulations to address complex conditions such as osteoarthritis, it is crucial to consider a wide range of bioactive components that can work synergistically. An integrative approach not only seeks to alleviate symptoms like pain and inflammation but also aims to slow disease progression by promoting tissue regeneration and reinforcing the structural integrity of the joints. In this context, key components include structural proteins like hydrolyzed collagen, glycosaminoglycans such as hyaluronic acid, and mineral supplements like calcium gluconate. Each of these elements can be adjusted in different concentrations to maximize their therapeutic effectiveness, offering a highly customizable treatment approach.

The flexibility to adjust these components across a variety of concentration ranges allows for tailored formulations that meet specific patient needs. For instance, hydrolyzed collagen can be included in concentrations ranging from 0.05% to 0.5%. Collagen plays a vital role in supporting the integrity of cartilage and connective tissues, which are often degraded in osteoarthritis. Hydrolyzed collagen, specifically, is more readily absorbed and utilized by the body, promoting chondrocyte proliferation and aiding in the repair of cartilage. By adjusting the concentration of hydrolyzed collagen, the formulation can provide varying levels of support for cartilage regeneration and joint repair, depending on the severity of the patient's condition. Lower concentrations of hydrolyzed collagen may be sufficient for patients in the early stages of osteoarthritis, where the primary goal is to preserve existing cartilage and prevent further deterioration. In contrast, higher concentrations may be more appropriate for patients with advanced joint degeneration, where the focus is on promoting new tissue growth and restoring joint function.

Similarly, glycosaminoglycans like hyaluronic acid can be incorporated at concentrations between 0.5% and 3%. Hyaluronic acid is essential for maintaining joint lubrication and reducing friction within the synovial space. For patients with mild symptoms or early osteoarthritis, a lower concentration of hyaluronic acid may provide sufficient lubrication and anti-inflammatory effects. However, in more advanced cases, higher concentrations may be necessary to restore the viscosity of the synovial fluid, protect against further cartilage breakdown, and improve overall joint mobility. Hyaluronic acid also has the added benefit of reducing pain and inflammation, making it an important component in the treatment of osteoarthritis.

Mineral supplements like calcium gluconate are another crucial part of the formulation, with concentrations ranging from 0.05% to 2%. Calcium gluconate plays a vital role in maintaining bone health, particularly in osteoarthritis patients who may suffer from reduced bone mineral density. Lower concentrations of calcium gluconate could be beneficial for maintaining bone strength in patients with early-stage osteoarthritis, while higher concentrations might be necessary for those at greater risk of bone loss and fractures due to long-term joint degeneration. By supporting the structural integrity of the bone matrix, calcium gluconate works in concert with hydrolyzed collagen and hyaluronic acid to offer comprehensive joint and bone protection.

This ability to customize the formulation by adjusting the ratios of these three critical components (hydrolyzed collagen, glycosaminoglycans, and mineral supplements) allows for a highly flexible and personalized therapeutic approach. The formulation can be tailored to address the specific needs of each patient, ensuring optimal therapeutic benefits while minimizing unnecessary exposure to higher concentrations of active ingredients.

In one preferred embodiment, the composition includes hyaluronic acid at a concentration of 1.50% per injection. In another embodiment, the composition includes hydrolyzed collagen at a concentration of 0.17% per injection. In a further embodiment, the composition includes calcium gluconate at a concentration of 0.33% per injection.

In one preferred embodiment, the composition, per injection, includes hyaluronic acid at a concentration of 1.50%, hydrolyzed collagen at a concentration of 0.17%, and calcium gluconate at a concentration of 0.33%.

In one embodiment, the composition may be formulated as: CBC (Cannabichromene): 1.0%; CBG (Cannabigerol): 1.8%; Glycosaminoglycans (Hyaluronic Acid): 1.5%; Structural Proteins (Hydrolyzed Collagen): 0.2%; Mineral Supplement (Calcium Gluconate): 0.5%.

In another embodiment, the composition may be formulated as: CBC (Cannabichromene): 0.8%; CBG (Cannabigerol): 2.0%; Glycosaminoglycans (Hyaluronic Acid): 1.5%; Structural Proteins (Hydrolyzed Collagen): 0.1%; Mineral Supplement (Calcium Gluconate): 1.0%.

In yet another embodiment, the formulation could include the following concentrations: CBC (Cannabichromene): 1.5%; CBG (Cannabigerol): 1.5%; Glycosaminoglycans (Hyaluronic Acid): 1.5%; Structural Proteins (Hydrolyzed Collagen): 0.3%; Mineral Supplement (Calcium Gluconate): 1.5%.

In a further embodiment, the composition may be designed with the following concentrations: CBC (Cannabichromene): 2.0%; CBG (Cannabigerol): 2.5%; Glycosaminoglycans (Hyaluronic Acid): 1.5%; Structural Proteins (Hydrolyzed Collagen): 0.05%; Mineral Supplement (Calcium Gluconate): 2.0%.

In another embodiment, the composition could include the following mixture: CBC (Cannabichromene): 0.5%; CBG (Cannabigerol): 1.0%; Glycosaminoglycans (Hyaluronic Acid): 1.5%; Structural Proteins (Hydrolyzed Collagen): 0.5%; Mineral Supplement (Calcium Gluconate): 0.3%.

Each of these embodiments provides flexibility in therapeutic design, allowing the composition to be tailored to different levels of osteoarthritis severity while ensuring that the key components work synergistically to enhance joint health, reduce inflammation, and promote tissue regeneration.

In one embodiment, the composition includes the following formulation: CBC (Cannabichromene): 0.83%; CBG (Cannabigerol): 1.5%; Glycosaminoglycans (Hyaluronic Acid): 1.5%; Structural Proteins (Hydrolyzed Collagen): 0.17%; Mineral Supplement (Calcium Gluconate): 0.33%.

In another embodiment, the composition may be adjusted as follows: CBC (Cannabichromene): 0.87%; CBG (Cannabigerol): 1.55%; Glycosaminoglycans (Hyaluronic Acid): 1.5%; Structural Proteins (Hydrolyzed Collagen): 0.17%; Mineral Supplement (Calcium Gluconate): 0.33%.

In yet another embodiment, the composition could include: CBC (Cannabichromene): 0.92%; CBG (Cannabigerol): 1.65%; Glycosaminoglycans (Hyaluronic Acid): 1.5%; Structural Proteins (Hydrolyzed Collagen): 0.17%; Mineral Supplement (Calcium Gluconate): 0.33%.

In a further embodiment, the composition may be formulated with the following configuration: CBC (Cannabichromene): 0.85%; CBG (Cannabigerol): 1.6%; Glycosaminoglycans (Hyaluronic Acid): 1.5%; Structural Proteins (Hydrolyzed Collagen): 0.17%; Mineral Supplement (Calcium Gluconate): 0.33%.

In another embodiment, the composition could include the following mixture: CBC (Cannabichromene): 0.89%; CBG (Cannabigerol): 1.52%; Glycosaminoglycans (Hyaluronic Acid): 1.5%; Structural Proteins (Hydrolyzed Collagen): 0.17% Mineral Supplement (Calcium Gluconate): 0.33%

These embodiments reflect subtle adjustments in the concentrations of CBC and CBG while maintaining constant levels of hyaluronic acid, hydrolyzed collagen, and calcium gluconate. This precision allows for highly tailored treatments that can be adjusted to meet the specific therapeutic goals and patient needs, ensuring a balanced and effective approach to managing osteoarthritis symptoms.

In one embodiment, the pharmaceutical composition may further include various excipients or carriers to enhance stability, bioavailability, and patient acceptability, supporting both the efficacy and shelf life of the formulation. These excipients are chosen based on their ability to facilitate the distribution of the active cannabinoids while maintaining the overall integrity of the composition. The selection of excipients plays a critical role in ensuring that the cannabinoids and other active ingredients, such as hyaluronic acid, hydrolyzed collagen, and calcium gluconate, are effectively delivered to the target tissue, remain stable during storage, and are well tolerated by patients.

Excipients may include solubilizers, emulsifiers, stabilizers, antioxidants, and preservatives, each serving a specific purpose within the formulation. Solubilizers, such as medium-chain triglycerides (MCT), can be used to enhance the solubility of hydrophobic cannabinoids like CBC and CBG, ensuring that they remain in solution and are readily available for absorption upon administration. Emulsifiers, such as lecithin or polysorbates, can help maintain a uniform distribution of the cannabinoids throughout the formulation, preventing phase separation and ensuring consistent dosing.

Stabilizers, including buffering agents, can be employed to maintain the pH of the composition within an optimal range, preventing the degradation of sensitive components like hyaluronic acid and ensuring that the formulation remains stable over time. Antioxidants, such as tocopherols (Vitamin E) or ascorbic acid, may be incorporated to protect the cannabinoids and other active ingredients from oxidative degradation, thereby extending the shelf life of the product and preserving its therapeutic potency.

Preservatives, such as benzyl alcohol or potassium sorbate, may be included to prevent microbial contamination, which is particularly important for formulations designed for parenteral administration. These preservatives help ensure the sterility and safety of the product, especially in multi-dose vials or formulations that may be stored for extended periods.

Together, these carefully selected excipients ensure that the pharmaceutical composition remains safe and effective, supporting optimal therapeutic outcomes and maximizing patient compliance across different administration routes, especially parenteral. The inclusion of excipients not only enhances the physical and chemical stability of the formulation but also improves patient experience by reducing irritation at the injection site, increasing comfort, and minimizing adverse reactions. By optimizing the solubility, stability, and delivery of the active ingredients, the excipients play a key role in enhancing the overall effectiveness and usability of the treatment.

In addition to parenteral formulations, excipients can also be tailored for other administration routes such as oral, sublingual, or topical, depending on the specific needs of the patient and the therapeutic goals. For instance, in oral formulations, flavoring agents or sweeteners may be added to improve palatability, while disintegrants can be included to ensure that tablets or capsules break down efficiently in the gastrointestinal tract, facilitating absorption. In topical formulations, excipients such as emollients or penetration enhancers may be used to improve the delivery of cannabinoids through the skin barrier, ensuring effective localized treatment.

Ultimately, the selection of excipients is a critical component of pharmaceutical formulation design, as they contribute not only to the stability and bioavailability of the active ingredients but also to the overall safety, efficacy, and patient acceptability of the product. This ensures that the therapeutic benefits of the composition are fully realized, providing patients with a reliable and effective treatment for conditions such as osteoarthritis.

In pharmaceutical formulations, particularly those intended for sensitive routes such as intra-articular administration, a significant portion of the composition often consists of excipients. These stabilizing, solubilizing, emulsifying, and lipid agents ensure that the active ingredients are delivered effectively, maintain stability over time, and minimize potential side effects. Typically, these excipients comprise a substantial percentage of the overall formulation, ranging anywhere from 70% and above, since their primary function is to support the active ingredients while ensuring the safety, efficacy, and bioavailability of the product.

In formulations where the active ingredients make up a smaller portion of the total product, the excipients take on an even more crucial role in maintaining the physical and chemical stability of the therapeutic compounds. This is particularly important for intra-articular formulations, which are administered directly into joint spaces. Any instability or improper solubility could reduce the therapeutic effectiveness or cause adverse reactions. Therefore, ensuring a high percentage of these excipients, typically above 70%, is vital to the successful delivery and prolonged shelf life of the composition.

In this embodiment, the stabilizing agents, solubilizing agents, emulsifying agents, and lipid agents in the intra-articular formulation are present at a concentration ranging from 95.08% to 97.27%. This high concentration guarantees the uniform distribution of the active ingredients, enhances bioavailability, and ensures the formulation's safety and efficacy for intra-articular delivery.

In further embodiments, these excipients could contain a variety of functional agents designed to enhance the formulation's stability, effectiveness, and overall patient experience. These excipients may include solubilizing agents, stabilizing agents, emulsifying agents, antioxidants, and preservatives, all working together to support the delivery of active ingredients like cannabinoids. Solubilizing agents are critical in formulations containing lipophilic compounds, such as cannabinoids, to ensure these ingredients are dissolved uniformly, thereby increasing their bioavailability. Stabilizing agents, on the other hand, are employed to maintain the formulation's integrity over time, preventing any degradation, separation, or loss of potency.

Emulsifying agents are particularly important in formulations where cannabinoids, often lipid-soluble, are combined with water-based substances. Emulsifiers create stable mixtures between oil and water phases, ensuring that cannabinoids remain evenly dispersed throughout the formulation. This prevents phase separation and ensures that the active ingredients are delivered in a consistent manner with each dose. This is especially vital in intra-articular formulations, where consistent therapeutic delivery directly impacts the efficacy of the treatment.

Antioxidants, such as tocopherols (Vitamin E), ascorbic acid, or other antioxidant compounds, are used to protect sensitive components like cannabinoids from oxidative degradation. Cannabinoids can be particularly susceptible to oxidation when exposed to air, light, or heat, leading to a loss of potency and therapeutic effectiveness. The inclusion of antioxidants helps to stabilize the formulation, extending its shelf life and preserving the bioactivity of the cannabinoids, ensuring they remain effective when administered.

Preservatives play a key role in maintaining the sterility and safety of the formulation, particularly in multi-use products or those intended for longer-term storage. For intra-articular formulations, where sterility is critical, preservatives such as benzyl alcohol or potassium sorbate help to prevent microbial contamination. This reduces the risk of infections or complications following injections, thereby ensuring that the formulation remains safe for repeated administration over time.

In further embodiments, these excipients could also include solubilizing and stabilizing agents, such as medium-chain triglycerides (MCTs), which are particularly effective at improving the solubility and absorption of cannabinoids. MCTs have excellent solubilizing properties for lipophilic substances, making them ideal for use in cannabinoid formulations. They help dissolve cannabinoids like CBC (cannabichromene) and CBG (cannabigerol) more effectively, ensuring that they remain in solution and are readily bioavailable when administered, especially in intra-articular dosage forms. This ensures that the active ingredients are delivered in a controlled, consistent manner, enhancing therapeutic efficacy while minimizing variability between doses.

The use of MCTs and other solubilizing/stabilizing agents is particularly important in intra-articular formulations, where consistency and precision in dosing are critical. Any separation or degradation of ingredients could result in uneven distribution of cannabinoids, potentially leading to suboptimal treatment outcomes. By incorporating excipients such as MCTs, the formulation remains homogeneous, ensuring that each injection delivers a precise dose of cannabinoids. This prevents phase separation or precipitation, ensuring the integrity of the formulation throughout its storage and use. These excipients, including solubilizing agents like medium-chain triglycerides, stabilizers, emulsifiers, antioxidants, and preservatives, collectively contribute to the overall effectiveness, stability, and safety of intra-articular formulations. They work together to ensure that the cannabinoids and other active ingredients are delivered consistently, remain stable over time, and retain their therapeutic potency. This comprehensive approach to formulation design not only enhances the bioavailability and absorption of the active ingredients but also ensures a safe and reliable product.

In one embodiment, the stabilizing and solubilizing agents for the injection may include medium-chain triglycerides (MCTs), which are particularly well-suited for enhancing the solubility and stability of lipophilic compounds such as cannabinoids. MCTs are known for their ability to create a uniform solution, which is critical for formulations where active ingredients like CBC and CBG need to be evenly distributed to ensure consistent dosing and therapeutic efficacy. The use of MCTs in these formulations helps prevent the separation or crystallization of cannabinoids, ensuring that the active components remain bioavailable and stable throughout the product's shelf life.

The concentration of medium-chain triglycerides in the injection typically ranges from 1% to 2%, with a preferred concentration of approximately 1.50%. This specific range has been selected to provide optimal solubility while maintaining the overall stability of the formulation. At concentrations within this range, MCTs effectively enhance the dispersion of cannabinoids, making them more readily absorbable when delivered via injection, particularly for intra-articular applications. In these scenarios, where precise and reliable delivery of the active ingredients is crucial, the solubilizing action of MCTs ensures that the cannabinoids are properly dissolved and can be absorbed by the surrounding tissues in the joint.

Moreover, medium-chain triglycerides have excellent bioavailability and are quickly metabolized by the body, which further supports their use as a solubilizing agent in injectable formulations. They enhance the transport of cannabinoids across cell membranes, aiding in faster absorption and ensuring that the therapeutic effects are realized more rapidly. This is particularly beneficial in the context of intra-articular injections, where timely relief from symptoms like pain and inflammation is desired.

Additionally, MCTs contribute to the overall stability of the formulation by reducing the risk of oxidation or degradation of the active cannabinoids. By maintaining the cannabinoids in a stable solution, MCTs help to prolong the shelf life of the product, ensuring that the therapeutic potency is retained over time. This stability is especially important for multi-dose formulations or those stored for extended periods, where maintaining the integrity of the active ingredients is critical for consistent therapeutic outcomes.

The 1.50% concentration of MCTs is an optimal balance for solubility, stability, and bioavailability. It ensures that the cannabinoids remain properly dispersed without oversaturating the formulation, which could lead to undesirable effects such as viscosity changes or difficulties in administration. At this preferred concentration, MCTs provide a smooth, consistent formulation that is easy to inject, ensuring that patients receive a uniform dose of the active ingredients with each administration.

Overall, the inclusion of medium-chain triglycerides as stabilizing and solubilizing agents in concentrations ranging from 1% to 2%, with a preferred concentration of 1.50%, plays a key role in enhancing the efficacy, safety, and reliability of cannabinoid-based injectable formulations. By improving solubility, preventing separation, and ensuring consistent absorption, MCTs help optimize the therapeutic delivery of cannabinoids, particularly in treatments requiring precise intra-articular administration.

Emulsifying agents, such as lecithin and polysorbates (e.g., polysorbate 80), are essential components in pharmaceutical formulations, particularly those containing lipophilic compounds like cannabinoids. These agents work to create stable, homogeneous mixtures in liquid formulations, ensuring that the active ingredients remain evenly distributed throughout the solution. This is especially important in intra-articular dosage forms, where precise and consistent delivery of cannabinoids is required to achieve the desired therapeutic effect. Without effective emulsifiers, cannabinoids could separate from the aqueous phase, leading to inconsistent dosing and potentially diminished therapeutic outcomes. Lecithin, derived from sources such as eggs or soy, is a natural emulsifier that forms micelles, encapsulating the hydrophobic cannabinoids and allowing them to remain stably dispersed in water-based formulations. Polysorbates, such as polysorbate 80, are synthetic emulsifiers that perform a similar function, stabilizing mixtures of oil and water to ensure homogeneity and prevent phase separation.

Antioxidants are another critical component of cannabinoid-based formulations, as cannabinoids are highly susceptible to oxidative degradation when exposed to environmental factors such as light, heat, air, and moisture. Cannabinoids, like CBC (cannabichromene) and CBG (cannabigerol), contain unsaturated bonds that make them particularly vulnerable to oxidation, which can lead to the breakdown of the active compounds, reducing their potency and efficacy. This degradation can significantly impact the therapeutic value of the formulation, as oxidized cannabinoids may no longer provide the desired pharmacological effects, such as pain relief or anti-inflammatory benefits.

Antioxidants such as ascorbic acid (Vitamin C) and tocopherols (Vitamin E) play a pivotal role in preserving the integrity of cannabinoid formulations by neutralizing reactive oxygen species (ROS) and free radicals that initiate oxidative degradation. Ascorbic acid, a water-soluble antioxidant, is particularly effective in aqueous environments, acting as a sacrificial electron donor to neutralize free radicals before they can damage the cannabinoids. Tocopherols, which are fat-soluble, are equally important in protecting the lipid components of the formulation, including the cannabinoids themselves. Tocopherols intercept and stabilize free radicals within the lipid phase, preventing oxidative chain reactions that could otherwise lead to the degradation of the entire formulation.

By preventing oxidation, these antioxidants extend the shelf life of cannabinoid formulations, ensuring that the active ingredients retain their potency and therapeutic effectiveness over time. This is particularly important in products intended for prolonged storage, such as multi-dose vials or formulations designed for long-term treatment regimens. Without adequate antioxidant protection, cannabinoids could lose a significant portion of their activity before the patient even begins using the product, leading to diminished clinical outcomes.

In addition to preserving the cannabinoids, antioxidants also help protect other excipients within the formulation that may be susceptible to oxidation. For example, lipid-based solubilizing agents, such as medium-chain triglycerides (MCTs), can also degrade when exposed to oxygen, leading to rancidity and compromising the stability of the entire formulation. The inclusion of antioxidants helps to safeguard these excipients as well, maintaining the overall quality and integrity of the product.

Further embodiments of the formulation may utilize a combination of antioxidants to create a more robust protective system. For instance, the synergistic use of both water-soluble and fat-soluble antioxidants (such as ascorbic acid and tocopherols) can offer comprehensive protection across both aqueous and lipid phases of the formulation. This ensures that the cannabinoids and other sensitive components are shielded from oxidative stress, regardless of their solubility characteristics. In some cases, chelating agents like ethylenediaminetetraacetic acid (EDTA) may also be included to bind metal ions that can catalyze oxidation, further enhancing the antioxidant protection.

The selection and concentration of antioxidants in cannabinoid formulations are carefully calibrated to provide optimal protection without compromising the stability or performance of the other components. Overuse of certain antioxidants could lead to instability in the formulation or undesirable interactions with other ingredients, while insufficient antioxidant levels may fail to adequately protect the cannabinoids from oxidative degradation. Therefore, achieving the right balance is essential for ensuring the long-term stability and effectiveness of the product.

The inclusion of antioxidants also contributes to the product's overall safety profile. Oxidative degradation not only reduces the potency of cannabinoids but may also produce harmful byproducts, such as peroxides or aldehydes, which could cause irritation or toxicity. By preventing oxidation, antioxidants help maintain the purity and safety of the formulation, ensuring that patients receive a high-quality product that is both effective and safe for use.

Ultimately, the strategic incorporation of antioxidants into cannabinoid-based formulations is essential for preserving the product's therapeutic value, extending its shelf life, and ensuring patient safety. This proactive approach to formulation design guarantees that the cannabinoids remain bioactive and effective, providing patients with reliable, long-lasting relief from conditions such as chronic pain, inflammation, and osteoarthritis, even after extended periods of storage.

In further embodiments, an emulsifying system may include additional agents such as sorbitan esters, egg lecithin, polysorbates, and poloxamers. These agents work synergistically to create a robust emulsification system that not only stabilizes the cannabinoids but also improves the overall performance and durability of the formulation. This is particularly critical in pharmaceutical formulations containing cannabinoids, which are hydrophobic and require precise emulsification to ensure consistent bioavailability and therapeutic efficacy.

Sorbitan esters, for example, are widely recognized for their excellent emulsifying properties in both water-in-oil (W/O) and oil-in-water (O/W) systems. These esters work by lowering the interfacial tension between the water and oil phases, promoting the formation of stable emulsions. In the context of cannabinoid formulations, sorbitan esters enable the creation of a homogenous mixture where lipophilic cannabinoids, such as CBC and CBG, are uniformly dispersed in aqueous environments. This is particularly important for formulations intended for injection, such as intra-articular applications, where precise and consistent cannabinoid delivery is essential. The emulsifying properties of sorbitan esters ensure that the cannabinoids remain stably suspended, preventing phase separation and ensuring that each dose contains the intended concentration of active ingredients.

Egg lecithin, a natural phospholipid, is another critical emulsifying agent in these formulations. It forms micelles and liposomes that encapsulate lipophilic compounds, such as cannabinoids, facilitating their dispersion in water-based solutions. Egg lecithin is particularly valuable in parenteral formulations due to its biocompatibility and ability to enhance the absorption of active ingredients. By incorporating egg lecithin into the emulsifying system, the formulation achieves better cannabinoid stability and bioavailability, as the lecithin helps protect the cannabinoids from degradation while improving their solubility in aqueous environments. This is especially important in maintaining the potency and therapeutic consistency of the formulation over time.

Polysorbates, such as polysorbate 80, are commonly used in pharmaceutical emulsions due to their ability to form stable oil-in-water emulsions. Polysorbates act as surfactants, reducing surface tension and facilitating the dispersion of cannabinoids in aqueous solutions. Their use in cannabinoid formulations helps maintain a uniform distribution of active ingredients, preventing the formation of clumps or aggregates. This is particularly important for ensuring that cannabinoids are evenly distributed throughout the formulation, allowing for consistent dosing with each administration. Polysorbates also enhance the bioavailability of cannabinoids by improving their solubility, making them more readily absorbed at the target site.

Poloxamers, also known as pluronic block copolymers, are versatile emulsifiers and stabilizers that can form micelles and enhance the solubility of hydrophobic compounds. Poloxamers have unique thermosensitive properties, meaning they can respond to temperature changes by transitioning between gel and liquid states, which is particularly useful for injectable formulations. In a cannabinoid-based formulation, poloxamers can form micelles that encapsulate the hydrophobic cannabinoids, ensuring that they remain stably suspended in aqueous environments. This encapsulation enhances the solubility and stability of the cannabinoids, protecting them from degradation and improving their bioavailability. Poloxamers are also valuable in preventing aggregation and sedimentation of cannabinoids, ensuring that the formulation remains homogeneous throughout its shelf life, even under varying storage conditions.

This combination of emulsifiers ensures that the formulation remains stable under a variety of conditions, such as fluctuations in temperature, agitation, or prolonged storage. Injectable formulations, in particular, require a high level of stability to ensure that the cannabinoids are delivered effectively and consistently with each dose. The inclusion of agents such as sorbitan esters, egg lecithin, polysorbates, and poloxamers creates a multi-faceted emulsifying system that provides both physical and chemical stability to the formulation. This is particularly important for intra-articular or intravenous injections, where inconsistent emulsification could lead to uneven dosing or reduced therapeutic efficacy.

Furthermore, these emulsifying agents also play a role in improving the overall patient experience by enhancing the formulation's stability and ease of administration. A stable emulsion reduces the risk of clogging or blockage in syringes or delivery devices, ensuring that the product can be administered smoothly and efficiently. Additionally, by maintaining the homogeneity of the formulation, the emulsifying system ensures that patients receive a consistent dose of cannabinoids, leading to more reliable therapeutic outcomes.

In summary, the incorporation of emulsifying agents such as sorbitan esters, egg lecithin, polysorbates, and poloxamers into cannabinoid-based formulations creates a robust system that stabilizes the cannabinoids, enhances solubility, and ensures the formulation's overall stability and performance. This combination of emulsifiers is particularly crucial for injectable formulations, where maintaining homogeneity and preventing phase separation are essential for ensuring consistent therapeutic delivery and optimal patient outcomes. The versatility of these emulsifying agents allows for a formulation that remains stable under various conditions, providing a reliable and effective solution for delivering cannabinoids in clinical applications.

In one specific embodiment, the emulsifier system comprises egg lecithin at a concentration ranging from 0.3% to 2%. Egg lecithin is a natural emulsifier known for its ability to create stable emulsions, particularly in formulations containing both hydrophilic and lipophilic components. At these concentrations, lecithin helps to ensure that the cannabinoids remain evenly distributed throughout the formulation, preventing phase separation and ensuring that each dose contains the intended amount of active ingredient. This is particularly critical in intra-articular injections, where inconsistent dosing could lead to suboptimal therapeutic effects or even treatment failure. By maintaining a homogenous mixture, lecithin ensures that each injection delivers a consistent and effective dose of cannabinoids, supporting the overall efficacy of the treatment.

The combined use of emulsifying agents like lecithin, polysorbates, and poloxamers, along with antioxidants such as ascorbic acid and tocopherols, ensures that the cannabinoid formulation remains stable, potent, and effective over time. This approach not only prevents physical separation and degradation of the cannabinoids but also enhances the solubility and bioavailability of the active ingredients, allowing for more consistent therapeutic delivery. This is especially important in parenteral dosage forms, where the precision of the formulation directly impacts the patient's clinical outcomes. By incorporating these excipients into the formulation, manufacturers can create a stable and effective treatment that meets the highest standards of safety and efficacy.

Preservatives, such as benzyl alcohol and potassium sorbate, play a crucial role in extending the shelf life of pharmaceutical formulations by preventing microbial contamination. This is particularly important in semi-solid and liquid forms, which are more susceptible to bacterial, fungal, and mold growth due to their higher moisture content. In multi-use containers, or in formulations designed for prolonged storage, preservatives ensure that the formulation remains sterile and safe for use over extended periods. Benzyl alcohol, for example, is widely used in injectable and topical formulations due to its antimicrobial properties and compatibility with various other ingredients. It is effective against a broad spectrum of bacteria, making it an ideal choice for formulations that need to remain free of contaminants during repeated use.

Potassium sorbate is another preservative commonly used in liquid and semi-solid formulations. It is particularly effective in preventing the growth of molds and yeasts, which can proliferate in water-containing formulations. Its use is favored for its relatively low toxicity and its ability to maintain the integrity of the formulation without negatively impacting the active ingredients. By incorporating preservatives like benzyl alcohol and potassium sorbate, manufacturers can ensure that the formulation remains free of microbial contamination.

In some embodiments, other excipients such as buffering agents and chelating agents may also be included to enhance the stability of the formulation. Buffering agents, such as citric acid or sodium citrate, are critical for maintaining the pH of the formulation within an optimal range. Cannabinoid formulations, like many pharmaceutical products, can be sensitive to fluctuations in pH, which can lead to degradation of the active ingredients or destabilization of the emulsion. Citric acid, for example, helps to stabilize the pH by acting as a weak acid buffer, ensuring that the formulation remains within a pH range that preserves the integrity of the cannabinoids and other active components. Maintaining a stable pH also contributes to the overall physical and chemical stability of the formulation, reducing the risk of degradation or separation over time.

Chelating agents, such as ethylenediaminetetraacetic acid (EDTA), are included in some formulations to prevent metal-induced oxidation. Trace metal ions, which may be introduced during the manufacturing process or through the use of certain packaging materials, can act as catalysts for oxidation, leading to the degradation of cannabinoids and other sensitive ingredients. Oxidation can result in a loss of potency, discoloration, or the formation of harmful byproducts. EDTA binds to these metal ions, effectively neutralizing their catalytic activity and preventing oxidation. By including chelating agents like EDTA, the formulation's stability is enhanced, prolonging its shelf life and ensuring that the active ingredients remain potent and effective throughout the product's intended use.

These stabilizing excipients work in concert to create a robust formulation that is resistant to physical, chemical, and microbial degradation. Preservatives like benzyl alcohol and potassium sorbate protect the formulation from microbial contamination, ensuring its safety for the patient. Buffering agents like citric acid maintain an optimal pH environment, preserving the integrity of the cannabinoids and other active ingredients. Chelating agents such as EDTA prevent metal-induced oxidation, safeguarding the formulation from degradation that could compromise its therapeutic efficacy.

In formulations intended for long-term storage or repeated use, this combination of preservatives, buffering agents, and chelating agents is essential to maintaining the product's stability. Preservatives ensure that microbial contamination does not compromise the formulation, while buffering and chelating agents maintain the chemical stability of the active ingredients. This is particularly important in liquid and semi-solid formulations, where the potential for degradation is higher due to the presence of water and other reactive components. By incorporating these excipients, the formulation remains safe, stable, and effective for its entire shelf life, providing patients with a reliable treatment option.

In summary, the use of preservatives such as benzyl alcohol and potassium sorbate extends the shelf life of cannabinoid formulations by preventing microbial growth. When combined with buffering agents like citric acid and chelating agents like EDTA, the overall stability of the formulation is significantly enhanced. These excipients work together to ensure that the formulation remains both physically and chemically stable, preserving the potency and efficacy of the active ingredients and providing patients with a safe and effective therapeutic product over an extended period.

In further embodiments, the antioxidants may include but are not limited to, vitamin E, ascorbic acid, butylated hydroxyanisole, or combinations thereof. These antioxidants are selected to stabilize the formulation by protecting cannabinoids from oxidative degradation, thereby preserving potency and extending shelf life. In certain embodiments, Vitamin E is included at a concentration ranging from 0.03% to 0.7%. Vitamin E acts as a protective antioxidant to prevent cannabinoid breakdown and enhances its absorption and bioavailability by supporting the integrity of lipid-based carriers within the formulation. The incorporation of antioxidants like Vitamin E and Vitamin C serves to maintain the therapeutic effectiveness of the cannabinoids over time.

In these embodiments, the composition may incorporate agents for pH regulation such as Sodium Hydroxide or Hydrochloric Acid in quantities sufficient to precisely adjust and maintain the pH within an optimal range. pH regulation is a critical factor in ensuring the stability, solubility, and bioavailability of the active ingredients, particularly in complex pharmaceutical formulations. Cannabinoid-based formulations, for example, may be sensitive to variations in pH, as fluctuations can lead to degradation of the active cannabinoids, impacting their potency and therapeutic effectiveness. Moreover, maintaining the correct pH is essential for preserving the integrity of other excipients and preventing undesirable interactions between components that could compromise the formulation's stability.

Sodium Hydroxide (NaOH) and Hydrochloric Acid (HCl) are commonly used for pH adjustments due to their effectiveness and compatibility with a wide range of pharmaceutical ingredients. Sodium Hydroxide, a strong base, is typically added to formulations to raise the pH when the mixture becomes too acidic. It neutralizes excess acidity, ensuring that the pH remains within a target range that protects the stability of both the cannabinoids and the excipients. Conversely, Hydrochloric Acid, a strong acid, is added to lower the pH when the formulation becomes too alkaline. The precise control of pH is especially important in aqueous formulations or those designed for parenteral administration, such as intra-articular injections, where even small deviations in pH can lead to discomfort, irritation, or reduced efficacy.

By incorporating these pH-adjusting agents, the formulation is able to achieve a balanced pH that optimizes the solubility and stability of the active ingredients. For instance, cannabinoids like CBC and CBG may exhibit improved solubility and absorption at specific pH levels, and controlling the pH ensures that these compounds remain bioavailable for therapeutic action. Furthermore, pH regulation can also prevent precipitation of the cannabinoids or other active components, which is crucial for ensuring consistent dosing and uniformity in liquid formulations.

In addition to improving the solubility of the active ingredients, pH regulation also plays a vital role in enhancing the safety and tolerability of the formulation. For products administered via injection, such as intra-articular or intravenous formulations, maintaining a pH that is compatible with the body's physiological environment is essential for reducing irritation and minimizing the risk of adverse reactions. Solutions with pH levels that deviate too far from the physiological norm can cause pain, tissue irritation, or even damage at the injection site. By adjusting the pH to match the body's natural pH (typically around 7.4), agents like Sodium Hydroxide and Hydrochloric Acid help to ensure that the formulation is both safe and comfortable for the patient.

Further, pH regulation can influence the chemical stability of the formulation, preventing reactions that could degrade the active ingredients. For example, some cannabinoids are prone to oxidation or hydrolysis at certain pH levels, and without proper pH control, the formulation could lose potency over time. Sodium Hydroxide and Hydrochloric Acid, when used appropriately, create a stable environment that prevents these degradative processes, thus extending the shelf life of the product and ensuring consistent therapeutic effects throughout its intended duration of use.

Moreover, in formulations that include other sensitive excipients, such as antioxidants, emulsifiers, or solubilizers, maintaining the right pH is essential to preserving their function. Many of these agents have pH-dependent stability profiles, and operating outside of their optimal pH range can lead to diminished performance or even the breakdown of the excipients. By adjusting the pH with agents like Sodium Hydroxide or Hydrochloric Acid, the formulation can remain stable and effective, ensuring that all components work harmoniously to deliver the intended therapeutic benefits.

In some embodiments, the pH-adjusting agents may be added in minimal quantities sufficient to achieve the desired pH without adversely affecting the other components of the formulation. These agents are typically introduced during the final stages of the manufacturing process, allowing for precise titration to achieve the target pH. This flexibility allows manufacturers to make minor adjustments in response to batch-specific variations, ensuring that each production run meets the desired specifications for stability, safety, and efficacy.

Overall, the incorporation of pH-regulating agents such as Sodium Hydroxide and Hydrochloric Acid is a vital step in the development of stable and effective pharmaceutical formulations. By carefully controlling the pH, these agents help to optimize the solubility, stability, and safety of the active ingredients, ensuring that the final product remains potent, reliable, and well-tolerated by patients across a range of therapeutic applications.

In further embodiments, the composition may incorporate an aqueous phase vehicle, which serves as a critical component for balancing the overall formulation and enhancing the solubility of the active ingredients. The aqueous phase vehicle, typically comprising water for injection (WFI), is present at a concentration ranging from 93% to 96%, providing the primary medium in which the other ingredients, including cannabinoids, are dissolved or dispersed. The use of water as the primary vehicle is particularly advantageous in pharmaceutical formulations, as it provides a neutral and biocompatible environment that is well-tolerated by the body, making it ideal for parenteral, oral, or topical administration.

The aqueous phase vehicle plays a crucial role in maintaining the physical and chemical stability of the formulation. In liquid or semi-liquid formulations, especially those intended for injection or topical use, water acts as a solvent that dissolves hydrophilic components and helps disperse hydrophobic substances, such as cannabinoids. This balanced environment prevents phase separation and ensures that the active ingredients remain uniformly distributed throughout the formulation. Consistent distribution is essential in ensuring that each dose contains the correct amount of active ingredients, thereby supporting reliable therapeutic efficacy across each administration. Without a properly balanced aqueous phase, the formulation could suffer from uneven dispersion of ingredients, leading to variability in dosing and potentially reducing the effectiveness of the treatment.

Furthermore, the aqueous phase vehicle significantly enhances the solubility of active ingredients. In formulations containing cannabinoids like CBC (cannabichromene) or CBG (cannabigerol), which are inherently lipophilic and exhibit poor water solubility, the presence of an aqueous phase vehicle helps facilitate their delivery when paired with appropriate solubilizing agents or emulsifiers. When cannabinoids are combined with an aqueous carrier, particularly in conjunction with solubilizers or emulsifying agents, their bioavailability can be significantly improved. This is especially important for water-based formulations, where the goal is to enhance the absorption of cannabinoids into the bloodstream or across biological membranes. Improved solubility and absorption ultimately translate into more effective therapeutic outcomes, as the active ingredients can reach their target sites more efficiently.

The use of water as an aqueous phase vehicle also helps to improve the consistency of dosage delivery. In injectable formulations, for example, a water-based carrier ensures that the cannabinoids and other active ingredients are consistently suspended in a fluid medium that is easy to administer. This facilitates smooth and precise injections, reducing the likelihood of inconsistencies that could arise from clumping or precipitation of active ingredients. Consistent delivery is essential in applications such as intra-articular injections, where precise control over the dosage is critical to achieving the desired therapeutic effect.

Moreover, an aqueous phase vehicle supports patient comfort and tolerability. Water for injection is a biocompatible carrier that mimics the natural aqueous environment of the human body. By using water as the primary vehicle, formulations are less likely to cause irritation or discomfort upon administration. This is particularly important in sensitive delivery routes such as parenteral or intra-articular injections, where the composition must be as non-irritating and physiologically compatible as possible. An aqueous-based vehicle also contributes to the overall safety of the formulation, as it provides a clean, inert medium that is free from potentially harmful additives or solvents.

The incorporation of an aqueous phase vehicle at a concentration ranging from 93% to 96% also helps to optimize the viscosity and fluidity of the formulation. For parenteral and intra-articular formulations, maintaining a proper viscosity is critical for ease of administration and ensuring that the solution flows smoothly through needles or delivery devices. If the formulation is too viscous, it may be difficult to inject or apply, while if it is too fluid, it may not remain in place long enough to exert its therapeutic effects. The water-based vehicle provides the necessary balance, ensuring that the formulation has the right consistency for its intended use while supporting efficient absorption and distribution of the active ingredients.

In addition to its functional roles in stability and solubility, the aqueous phase vehicle also enhances the overall formulation's stability. Water, when properly purified for injection, minimizes the risk of contamination and degradation of sensitive components. It helps maintain a controlled environment that protects the cannabinoids and other excipients from environmental stressors such as heat or light. Moreover, water for injection is processed to meet stringent standards of sterility, ensuring that the formulation remains free from microbial contamination during production and throughout its shelf life. This contributes to the longevity of the formulation, ensuring that it remains safe and effective over time.

The inclusion of an aqueous phase vehicle, comprising 93% to 96% water for injection, is vital to the overall success of the formulation. It serves multiple roles in enhancing the solubility and bioavailability of cannabinoids, maintaining the stability and uniformity of the formulation, and ensuring patient comfort during administration. By providing a neutral and biocompatible medium, the aqueous phase vehicle facilitates consistent and reliable dosage delivery, contributing to the effectiveness and safety of cannabinoid-based treatments, particularly in parenteral or injectable applications.

In some embodiments, the composition includes cannabinoids, structural components of the extracellular matrix, and calcium compounds as active principles. In a further embodiment, the composition comprises cannabinoids ranging from 2.33% to 2.57%, structural components of extracellular matrix ranging from 1.5% to 2%, and calcium supplements ranging from 0.3% to 0.4% per injection. In a preferred embodiment, the composition comprises cannabinoids such as CBC and CBG, structural proteins such as hydrolyzed collagen, glycosaminoglycans such as hyaluronic acid, and calcium compounds such as calcium gluconate. In a further preferred embodiment, the composition comprises CBC 0.83%, CBG 1.50%, hyaluronic acid 1.50%, hydrolyzed collagen 0.17%, and calcium gluconate 0.33%.

In another embodiment, the composition additionally contains stabilizing agents and antioxidant agents. In a further embodiment, the stabilizing agents range from 0.3% to 2% and the antioxidants from 0.03% to 0.7%. In a preferred embodiment, the composition comprises stabilizing agents such as Medium-chain Triglycerides and Egg Lecithin, and antioxidants such as vitamin E. In a further preferred embodiment, the composition comprises Medium-chain Triglycerides at 1.50%, Egg Lecithin at 0.5%, and vitamin E at 0.05%.

In some embodiments, this pharmaceutical composition is designed for multiple routes of administration and in various pharmaceutical forms, providing a versatile framework for delivering cannabinoids and other active ingredients in a manner that maximizes therapeutic efficacy while catering to patient needs and preferences. The ability to adapt the formulation for different administration routes not only increases its applicability across a broad spectrum of medical conditions but also allows for customization in terms of dosage, onset of action, and patient comfort.

In a further embodiment, the routes of administration may include oral, sublingual, topical, inhalation, parenteral, and intravaginal, each offering distinct advantages depending on the target condition and the desired therapeutic effect. Oral administration, through forms such as tablets, capsules, or syrups, allows for systemic distribution of the active ingredients, typically with a slower onset but longer duration of action. Sublingual administration, on the other hand, offers a faster onset by bypassing the digestive system and delivering the active ingredients directly into the bloodstream through the mucous membranes under the tongue. Topical formulations, such as creams or gels, are ideal for localized treatment of conditions such as inflammation or pain, providing direct relief to the affected area without systemic exposure.

Inhalation, often through sprays or aerosols, allows for rapid absorption of cannabinoids via the respiratory system, making it suitable for conditions that require fast relief, such as acute pain or anxiety. Parenteral routes, which include injections (such as intravenous, intramuscular, or intra-articular), provide precise and immediate delivery of the active ingredients into the bloodstream or directly into the joint space, making it highly effective for conditions that require quick and targeted intervention, such as osteoarthritis or other inflammatory disorders. Intravaginal administration through ovules or suppositories offers targeted delivery for gynecological conditions, providing localized relief while minimizing systemic side effects.

In terms of pharmaceutical forms, this composition is highly adaptable. It may be formulated as tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. Each form provides unique advantages in terms of stability, patient compliance, and the delivery profile of the active ingredients. For instance, sublingual films or tablets provide a discreet and convenient method of administration that bypasses first-pass metabolism, while gummies and caramels offer a patient-friendly option that enhances compliance, particularly in pediatric or geriatric populations.

In a preferred embodiment, this pharmaceutical composition is intended for intra-articular administration and presented in the form of an injectable solution or suspension. This route is particularly advantageous for delivering cannabinoids directly into the joint space, where they can exert potent anti-inflammatory and analgesic effects. Intra-articular injections are commonly used for the treatment of osteoarthritis and other joint-related conditions, where direct access to the site of inflammation allows for localized relief without systemic side effects.

Furthermore, the concept of the Doctrine of Equivalents is highly relevant when discussing the flexibility and adaptability of this pharmaceutical composition across different forms and routes of administration. The doctrine allows for patent protection not only on the specific embodiments of a composition but also on equivalent variations that achieve the same function in substantially the same way, with the same result. For instance, while a composition may be specifically designed for intra-articular administration, an equivalent formulation delivered via a different route, such as oral or sublingual, may also fall within the scope of the patent if it achieves the same therapeutic effect using equivalent ingredients or methods. This legal principle ensures that minor modifications in formulation or method of delivery do not circumvent the intellectual property protections granted to the original invention.

In applying the Doctrine of Equivalents to this pharmaceutical composition, any adjustments made to the concentration of active ingredients, excipients, or the method of administration, whether shifting from oral to injectable forms, or modifying the composition for sublingual versus topical application, could still be considered within the bounds of the original invention, as long as the therapeutic outcomes remain comparable. This flexibility allows for the composition to be adapted for different patient populations, medical conditions, and delivery systems without losing the essence of its inventive contribution. For example, a formulation designed for intra-articular injection that is later adapted for oral delivery using an advanced bioavailability-enhancing technology could be seen as an equivalent under this doctrine, providing the same therapeutic benefits through a different route of administration.

In summary, the pharmaceutical composition's design for multiple routes of administration and a wide array of pharmaceutical forms showcases its versatility and potential for broad therapeutic application. The inclusion of the Doctrine of Equivalents further reinforces the composition's adaptability, ensuring that minor modifications or variations in the method of delivery are still protected, maintaining the composition's utility across various therapeutic contexts while safeguarding the innovation behind its development.

### Treatment Protocol:

Administration and Dosage Regimen: The treating physician must administer the injection into the intra-articular space of the affected knee or both knees, using ultrasound guidance to ensure precision. Prior to injection, local anesthesia, typically lidocaine, should be used to numb the area and enhance patient comfort during the procedure. The injection protocol involves a single administration every three months, continued for a duration of one year. This regimen is designed to provide sustained therapeutic effects while minimizing the need for frequent dosing, thus improving patient adherence and overall treatment outcomes.

In one embodiment, the composition may be tailored for multiple routes of administration beyond intra-articular injection, depending on the patient's condition and therapeutic goals. These administration forms may include oral, topical, or transdermal preparations, ensuring flexibility in treatment options. Additionally, the composition can be formulated in various pharmaceutical forms, such as solutions, emulsions, or nanoemulsions, to optimize bioavailability and patient compliance.

To further customize the therapeutic experience, the composition may also be developed with different controlled-release mechanisms, including immediate, sustained, or delayed-release formulations. Controlled-release options ensure that the active cannabinoids, such as CBC and CBG, are delivered over an extended period, maximizing efficacy while reducing the need for repeated dosing. This flexibility allows healthcare providers to tailor the treatment regimen based on the severity of the osteoarthritis, patient response, and any specific medical considerations.

Compositions of the disclosure can be presented in a wide range of pharmaceutical dosage forms, tailored to optimize patient compliance and enhance therapeutic efficacy across various treatment settings. These forms encompass solid, semisolid, liquid, and gaseous formulations, each specifically engineered to target distinct administration pathways, ensuring flexibility in treatment approaches and maximizing therapeutic potential.

In one embodiment, the phytocannabinoid-based pharmaceutical composition is designed to support multiple routes of administration, adapting to a variety of therapeutic applications. These routes may include, but are not limited to, oral, sublingual, topical, inhalation, parenteral, and intravaginal delivery methods. Each form offers flexibility in treatment and enables precise control over dosage and bioavailability, addressing diverse patient needs and optimizing cannabinoid delivery to specific sites of action.

Oral formulations, such as capsules, tablets, or soft gels, offer a convenient and familiar administration route, especially suited for patients seeking ease of use and long-term management of chronic conditions. Sublingual forms, such as sprays or dissolvable films, provide a faster onset of action by bypassing gastrointestinal metabolism and allowing for quicker absorption directly into the bloodstream. Both oral and sublingual methods are particularly advantageous for systemic treatment, ensuring prolonged bioavailability of the active cannabinoids.

Topical formulations, including creams, gels, and transdermal patches, are specifically designed for localized treatment of inflammatory or neuropathic pain. These forms enable the cannabinoids to penetrate through the skin barrier, offering direct therapeutic action to target tissues without significant systemic absorption. Transdermal patches, in particular, allow for controlled, sustained release of cannabinoids over time, making them ideal for long-term pain management.

Inhalation, via aerosolized sprays or vaporized cannabinoid products, is an efficient route of administration for achieving rapid therapeutic effects. This method allows for immediate cannabinoid delivery through the pulmonary system, making it highly suitable for acute symptom relief in conditions such as severe pain or inflammation. The ability to deliver cannabinoids directly to the bloodstream via the lungs bypasses first-pass metabolism and provides near-instantaneous onset of action.

Parenteral formulations, designed for intra-articular, subcutaneous, intravenous, or intramuscular administration, offer precise and controlled delivery of cannabinoids, particularly in clinical settings requiring immediate therapeutic action. In one embodiment, the composition is formulated into injectable solutions or suspensions, which are ideal for scenarios where rapid absorption and targeted delivery are essential, such as in acute pain management or inflammation control. Intra-articular injections are particularly useful for localized treatment of joint-related conditions such as osteoarthritis. Administered directly into the joint space, these injections allow for high concentrations of cannabinoids to act on inflamed tissues, providing sustained relief from pain and inflammation. Intravenous formulations offer systemic cannabinoid delivery with immediate therapeutic effect, crucial in settings where fast action is required, such as acute pain episodes or inflammatory responses. Subcutaneous and intramuscular injections provide a slower, sustained release compared to intravenous, making them suitable for conditions that benefit from gradual cannabinoid absorption over time, offering prolonged relief from chronic symptoms.

Intravaginal and rectal applications, designed as suppositories or creams, are developed for direct application to mucosal membranes, allowing for both localized and systemic absorption of cannabinoids. This method is particularly effective for managing conditions such as pelvic pain, endometriosis, or localized inflammation while minimizing systemic side effects.

To further optimize therapeutic outcomes, the composition may be developed with controlled-release technologies, allowing for immediate, sustained, or delayed-release profiles depending on the patient's clinical needs. Controlled-release formulations are designed to provide a steady concentration of cannabinoids in the system over an extended period, reducing the frequency of dosing and improving overall patient adherence. Sustained-release technologies, such as microencapsulation or nanoparticle-based delivery systems, ensure that the active ingredients are gradually released, maintaining therapeutic levels without the need for frequent administrations. Delayed-release formulations can be tailored for conditions requiring time-specific delivery or localized action.

In further embodiments, the composition may utilize nanotechnology-based delivery systems to enhance bioavailability and target specific tissues more effectively. Nanoparticles, nanoemulsions, or liposomes can be employed to protect cannabinoids from degradation and increase their absorption through biological barriers, such as the blood-brain barrier or dermal layers. These advanced delivery systems ensure that cannabinoids are more efficiently absorbed and retained in therapeutic concentrations for a longer duration, providing enhanced relief with minimal side effects.

By offering a range of flexible dosage forms and administration routes, the phytocannabinoid-based composition ensures precise control over dosage, enhanced patient compliance, and tailored treatment regimens to meet the varying needs of patients across a wide spectrum of medical conditions.

Compositions of the disclosure may be presented in any pharmaceutical dosage forms such as solid, semisolid, liquid and gas forms. In one embodiment the pharmaceutical dosage form may be tablets, capsules, powder, dusting powder, or other related solid forms. In another embodiment, the pharmaceutical dosage form may be cream paste, gel, suppositories or other related semi solid forms. In another embodiment, the pharmaceutical dosage form may be syrup, solution, emulsion, suspension, or other related liquid forms. In another embodiment, the pharmaceutical dosage form may be inhaler, aerosols or other related gas forms.

In a further preferred embodiment, the pharmaceutical form may be intra-articular injection. In one embodiment, the pharmaceutical dosage form may be a parenteral form, such as intra articular or dusting powders, which are convenient for parenteral administration and suitable for controlled-release formulations. These parenteral forms allow precise dosing and are often preferred for their stability and ease of transport and storage.

In another embodiment, the composition may be formulated as a semisolid, such as creams, pastes, gels, or suppositories, which provide localized or systemic absorption.

In another embodiment, the pharmaceutical dosage form may be a liquid, including syrups, solutions, emulsions, or suspensions, which are suitable for oral, sublingual, or parenteral administration. Liquid formulations offer flexibility in dosing and rapid absorption, making them ideal for individuals requiring swift symptom relief or for patients who have difficulty swallowing solid forms.

In an additional embodiment, the composition may be administered in a gaseous form, such as through inhalers or aerosols, which are suitable for pulmonary delivery. This form allows for rapid absorption of cannabinoids via the respiratory tract, providing an efficient delivery method for systemic effects with fast onset, useful in managing acute symptoms.

In a further preferred embodiment, the pharmaceutical form may be intra-articular injection. This formulation provides a precise and controlled parenteral dosing option. These preferred forms enhance bioavailability and provide versatile options for both systemic and localized therapeutic effects tailored to the specific needs of osteoarthritis patients.

In some modalities, the pharmaceutical forms may offer immediate-release, sustained-release, or controlled-release options, allowing for precise regulation of cannabinoid bioavailability over time. Immediate-release formulations are designed to provide rapid relief from acute symptoms, while sustained-release forms gradually release the active ingredients over a prolonged period, maintaining stable therapeutic levels and reducing dosing frequency. Controlled-release options, on the other hand, allow for more specific delivery by maintaining a constant release rate, optimizing cannabinoid levels in the body for effective management of chronic symptoms and promoting better patient adherence. These release mechanisms enable the formulation to be tailored to various therapeutic needs, from rapid relief to long-term control of osteoarthritis symptoms.

### Method of Treatment

The method of treatment is presented as an injectable nanoemulsion for intra-articular administration, combining 25 mg of Cannabichromene (CBC) and 45 mg of Cannabigerol (CBG), two phytocannabinoids with notable pharmacological properties. It includes 45 mg of HA for joint lubrication, 5 mg of hydrolyzed collagen to facilitate the biosynthesis of new cartilage tissue, and 10 mg of calcium gluconate, which are key nutrients for joint health.

The method of treatment constitutes an intra-articular injection formulated for knee osteoarthritis that targets inflammation, cartilage protection, and pain relief. Cannabinoids like CBC and CBG primarily interact with CB1 and CB2 receptors, with CB2 activation playing a key role in modulating inflammation and reducing pain. CBC reduces inflammatory markers by inhibiting the NF-κB and MAPK pathways, while CBG offers anti-inflammatory and antioxidant effects by lowering pro-inflammatory cytokines (like IL-1β and IFN-γ) and enhancing antioxidant defenses through increased superoxide dismutase (SOD) activity. These cannabinoids help decrease oxidative stress and protect joint tissue from further degradation.

Hyaluronic acid (HA) helps restore the viscosity and elasticity of synovial fluid, lubricating the joint and promoting better cartilage function. It also interacts with CD44 receptors to reduce pro-inflammatory cytokines and enzymes, enhancing joint protection and reducing synovial inflammation. Collagen complements HA by promoting cartilage regeneration, stimulating hyaluronic acid production, and reducing matrix-degrading enzymes like MMPs and ADAMTS, which are often elevated in osteoarthritis. Together, these components work to slow the progression of cartilage degradation, reduce pain and inflammation, and improve joint mobility and overall knee function in osteoarthritis patients.

In this embodiment, these concentrations are designed to deliver effective relief for osteoarthritis symptoms such as persistent knee pain, stiffness, reduced function, and in more advanced cases, crepitus, restricted joint motion, and bone enlargement. The method of treatment is designed for targeted delivery of these bioactive compounds to the application site, offering an innovative option for managing OA.

In this embodiment the environment must be sterile, reflecting the critical importance of maintaining asepsis throughout the procedure to minimize the risk of infection and maximize the success rate of the injection.

In some embodiments the sterile environment is paramount for the procedure, encompassing not only the physical space but also the personal protective equipment worn by the healthcare professional. This includes the use of sterile gloves, which serve as a barrier between the practitioner and the patient, preventing the transmission of pathogens.

In some embodiments the sterile needle, typically a 21-Gauge×1.5-inch needle, is another critical element of the sterile environment, ensuring that the injection is delivered accurately and safely.

In one embodiment the patient's positioning plays a significant role in the procedure's success. For different approaches, the patient can be seated or lying supine with the knee flexed at approximately 90 degrees. This positioning facilitates better exposure of the intra-articular surface, simplifying the insertion of the needle into the joint space.

In this embodiment, the choice of approach, whether suprapatellar, lateral, mediolateral, superolateral infrapatellar, medial, superomedial infrapatellar, or anterior (less commonly used), is guided by the path of least obstruction and maximum access to the synovial cavity, while considering the patient's bony anatomy.

In some embodiments, ultrasound-guided puncture allows us to access the articular space of the knee with a single puncture and without causing any friction on the articular cartilage. Prior to the administration of the injection, comprehensive information regarding the subject's medical history, current and past treatments, previous procedures, emergence of new symptoms, and signs of inflammation or infection must be collected. It is imperative that a clinical assessment confirms the absence of knee swelling, which could pose a risk of infection.

In this embodiment, pre-injection skin preparation must adhere to strict aseptic protocols, involving the application of cleaning materials (i.e. gauze) into one or two layers of povidone iodine followed by isopropyl alcohol 70%, with the healthcare provider utilizing sterile gloves.

In this embodiment, the use of disinfectants containing quaternary ammonium salts for skin preparation is contraindicated due to the potential precipitation of hyaluronan in their presence and can produce harmful reactions on skin and mucous membranes.

In this embodiment, local anesthesia, achieved through a lidocaine 2% injection, is administered prior to the intra-articular injection.

In some embodiments, the physician's selected approach involves introducing a 21-gauge needle into the joint space, followed by an aspiration attempt to withdraw synovial fluid. This step serves to confirm precise needle placement within the joint cavity, facilitating the evacuation of synovial fluid and any associated effusion, thereby enhancing diagnostic accuracy and therapeutic outcomes.

In one embodiment, the injection is then performed without resistance or discomfort. Upon completion of the injection, the needle is withdrawn and discarded in a sharps and biological container. Subsequently, the knee is gently moved through flexion and extension movements to assist in the distribution of the injected substance.

In these embodiments immediately following the injection, patients are advised to refrain from engaging in strenuous activities that place excessive stress on the knee for 48-72 hours. This includes high-impact exercises like jogging, heavy lifting, or extended periods of standing.

### Manufacturing Method:

In some embodiments, the fabrication of the formulation necessitates adherence to stringent standards within a sterile environment. All laboratory equipment utilized must undergo thorough sterilization processes to ensure the highest degree of purity and safety.

In other embodiments, the compounds identified within the formulation must be prepared in injectable solution formats, with the active ingredients formulated as nanoemulsions created by ultrasonic cavitation to optimize bioavailability and efficacy.

In one embodiment, nanoemulsions are a nano-sized emulsion system which offer advantages over conventional emulsions, such as unique functional properties, higher stability, transparency. Ultrasonic or acoustic cavitation generates high shear forces, which provide the energy needed to disintegrate large droplets down to nanometer-sized droplets. In this way, two or more liquid phases are mixed into a uniform sub micrometer or nanometer emulsion.

In other embodiments, nanoemulsions, also known as nanometric-sized emulsions, are fine water-in-oil (w/o) and oil-in-water (o/w) dispersions of two immiscible fluids, as opposed to the milky-white hue concomitant with coarse dispersion. These 20-200 nm droplets are stabilized by adding the appropriate amphiphilic emulsifiers or emulsifiers. Consequently, nanoemulsions are also known as mini-emulsions. Due to kinetic stability, nanoemulsions (NE) are stable on heterogeneous systems, in contrast to microemulsions (ME). Although nanoemulsions are unique due to their extended physical constancy and are also known as “potential thermodynamic stability,” they do not appear to aggregate or flocculate.

In one embodiment, an emulsion is a biphasic system in which one phase is intimately dispersed in the other phase in the form of minute droplets ranging in diameter from 0.1 to 100 μm. It is a thermodynamically unstable system, which can be stabilized by the presence of an emulsifying agent (emulgent or emulsifier).

The dispersed phase is also known as internal phase or the discontinuous phase while the outer phase is called dispersion medium, external phase or continuous phase. The emulsifying agent is also known as intermediate or interphase. The term ‘nanoemulsion’ also refers to a miniemulsion which is fine oil/water or water/oil dispersion stabilized by an interfacial film of surfactant molecule having droplet size range 20-600 nm. Because of their small size, nanoemulsions are transparent. There are three types of nanoemulsion which can be formed: (a) oil in water nanoemulsion in which oil is dispersed in the continuous aqueous phase, (b) water in oil nanoemulsion in which water droplets are dispersed in continuous oil phase, and (c) bi-continuous nanoemulsions.

In this embodiment, the treatment is formulated as an oil-in-water nanoemulsion, wherein the oil phase is dispersed within a continuous aqueous medium. This formulation leverages the hydrophobic character of cannabinoids, which are incorporated into the oil phase, alongside the hydrophilic attributes of the remaining active ingredients (hyaluronic acid, hydrolyzed collagen, calcium gluconate) which are seamlessly integrated into the water phase.

In various embodiments, nanoparticle technology offers a series of advantages for drug delivery, including high loading yield, combination therapy, controlled release, prolonged circulation, and targeted delivery.

In various embodiments, a myriad of nanoparticle-based drug delivery systems have been developed to improve therapeutic index of drugs by altering their pharmacokinetics and biodistribution profiles, resulting in nanomedicines for clinical treatment of various diseases.

In one embodiment, to ensure product sterility, a controlled sterilization method has been employed. In this embodiment, the sterilization process can be carried out using techniques that do not affect the integrity of cannabinoids, as these compounds are sensitive to thermal and photochemical degradation.

In this embodiment, the preferred sterilization process is filtration sterilization, which involves passing the cannabinoid-containing solution through a 0.22-micron filter, removing microbial particles without the need for heat or radiation, thus preventing the decomposition of cannabinoids.

In these preferred embodiments, the nanoemulsion is introduced into the syringe after a sterilization process, using a 0.22 μm filtering membrane under aseptic conditions.

In certain embodiment cases, a low-dose gamma irradiation sterilization process can also be employed, preferably in the range of 2 to 5 kGy, which is sufficient to eliminate microbiological contaminants without affecting the chemical structure of the cannabinoids. This procedure is applicable once the solution has been packaged in the syringe and hermetically sealed, ensuring sterility without exposing the formulation to high temperatures. In another embodiment, for some cannabinoids more sensitive to irradiation, sterilization by ethylene oxide (EtO) under controlled temperature and time conditions may be chosen. This method allows for device sterilization without compromising the integrity of the cannabinoids, provided that adequate post-ventilation is conducted to remove any residual EtO gas.

### EXAMPLES

### Pharmaceutical Compositions

The current example discloses a therapeutic agent aimed at treating osteoarthritis. This agent comprises a unique composition that utilizes nanoplatform containing cannabinoids, such as CBC and CBG. The composition is defined by designated percentage ranges of each to ensure effective administration.

The example describes a substance designed to treat osteoarthritis in patients. This substance comprises a composition that utilizes nanoplatform containing cannabinoids, such as CBC and CBG. Each compound's percentage range for administration is outlined to optimize therapeutic effects.

Particular amounts of cannabinoids: 25 mg of CBC, 45 mg of CBG per injection.

Intra articular injection: composition 1: 70 mg of cannabinoids per injection.

| Component | Quantity (mg) | Percentage (%) |
|---|---|---|
| CBC | 25 | 0.83 |
| CBG | 45 | 1.50 |
| Hyaluronic acid | 45 | 1.50 |
| Hydrolysate collagen | 5 | 0.17 |
| Calcium gluconate | 10 | 0.33 |
| Egg Lecithin | 15 | 0.50 |
| Alpha Tocopherol (Vitamin E) | 1.5 | 0.05 |
| Medium-chain Triglycerides | 45 | 1.50 |
| Sodium Hydroxide for pH adjustment | qs pH 6.0-7.0 | qs pH 6.0-7.0 |
| Hydrochloric Acid for pH adjustment | qs pH 6.0-7.0 | qs pH 6.0-7.0 |
| Water for injection | qs 3.00 mL | 95.22 |

Intra articular injection: composition 2: 77 mg of cannabinoids per injection.

| Component | Quantity (mg) | Percentage (%) |
|---|---|---|
| CBC | 27.5 | 0.92 |
| CBG | 49.5 | 1.65 |
| Hyaluronic acid | 45 | 1.50 |
| Hydrolysate collagen | 5 | 0.17 |
| Calcium gluconate | 10 | 0.33 |
| Egg Lecithin | 15 | 0.50 |
| Alpha Tocopherol (Vitamin E) | 1.5 | 0.05 |
| Medium-chain Triglycerides | 38 | 1.27 |
| Sodium Hydroxide for pH adjustment | qs pH 6.0-7.0 | qs pH 6.0-7.0 |
| Hydrochloric Acid for pH adjustment | qs pH 6.0-7.0 | qs pH 6.0-7.0 |
| Water for injection | qs 3.00 mL | 93.03 |

### Nanoemulsion Process:

### Example 1

Oil phase: A quantity of 1.50 grams of medium-chain triglycerides (MCT) is added to a suitable container and heated to 40° C.-45° C. Subsequently, 1.5 grams of CBG. Immediately, 0.83 grams of CBC are added and 0.05 grams of the Vitamine E. The mixture is stirred at a speed of 500 revolutions per minute (rpm) until all components are fully integrated. The oil phase maintained the temperature between 40° and 45° C.

Aqueous phase: A quantity of 95.22 grams of Water for injection is added to a suitable container and heated to 40° C.-45° C. Subsequently, 0.5 grams of Egg Lecithin, 1.5 grams of hyaluronic acid, 0.17 grams of Hydrolysate collagen and dissolved. Immediately, 0.33 grams of Calcium gluconate are added. The mixture is stirred at a speed of 500 revolutions per minute (rpm) until all components are fully integrated.

Formation of the preliminary emulsion: Once both the oil and aqueous phases have reached the established temperature between 40° C. and 45° C., the oil phase is slowly and gradually added to the aqueous phase, under constant stirring. The mixture is stirred for a period of 30 minutes to ensure the formation of a homogeneous preliminary emulsion. Check and adjust the pH of the formulation from 6 to 7 with Sodium Hydroxide or Hydrochloric Acid.

Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 45° C.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Injection filling: Once the nanoemulsion has reached the appropriate temperature, the syringe is filled. The nanoemulsion is introduced into the syringe after a sterilization process, using a 0.22 μm filtering membrane under aseptic conditions. It is important to maintain a nitrogen atmosphere throughout manufacturing with nitrogen gas.

### Example 2

Oil phase: A quantity of 1.5 grams of medium-chain triglycerides (MCT) is added to a suitable container and heated to 40° C.-45° C. Subsequently, 1.65 grams of CBG. Immediately, 0.92 grams of CBC are added and 0.05 grams of the Vitamin E. The mixture is stirred at a speed of 500 revolutions per minute (rpm) until all components are fully integrated. The oil phase maintained the temperature between 40° C. and 45° C.

Aqueous phase: A quantity of 93.03 grams of Water for injection is added to a suitable container and heated to 40° C.-45° C. Subsequently, 0.5 grams of Egg Lecithin, 1.5 grams of hyaluronic acid, 0.17 grams of Hydrolysate collagen and dissolved. Immediately, 0.33 grams of Calcium gluconate are added. The mixture is stirred at a speed of 500 revolutions per minute (rpm) until all components are fully integrated.

Formation of the preliminary emulsion: Once both the oil and aqueous phases have reached the established temperature between 40° C. and 45° C., the oil phase is slowly and gradually added to the aqueous phase, under constant stirring. The mixture is stirred for a period of 30 minutes to ensure the formation of a homogeneous preliminary emulsion. Check and adjust the pH of the formulation from 6 to 7 with Sodium Hydroxide or Hydrochloric Acid.

Formation of the nanoemulsion: To reduce the droplet size in the preliminary emulsion to a range of 50 to 200 nanometers (nm), a high-pressure homogenizer is used. The homogenization process is carried out by applying pressures between 1000 and 1500 bar, over 3 to 5 cycles of homogenization. During each cycle, the emulsion is forced through a narrow valve at high speed, generating shear forces that reduce the droplet size. It is crucial to monitor the temperature of the emulsion throughout the process, keeping it between 40° C. and 45° C., using a cooling system if necessary, to preserve the stability of the active ingredients

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Injection filling: Once the nanoemulsion has reached the appropriate temperature, the syringe is filled. The nanoemulsion is introduced into the syringe after a sterilization process, using a 0.22 μm filtering membrane under aseptic conditions. It is important to maintain a nitrogen atmosphere throughout manufacturing with nitrogen gas.

As an example, these are some embodiments of the disclosure:

A composition for treatment of osteoarthritis, comprising:
a) an active principle comprising at least two cannabinoid;
b) a second active principle comprising at least one or more additional active agents including structural proteins, glycosaminoglycans, mineral supplement, or combinations thereof; and
optionally c) a stabilizing/solubilizing agent.
wherein the composition is suitable for intra articular administration.

In one embodiment, in the preceding composition, the cannabinoid comprises one or more of the following: cannabichromene (CBC), cannabigerol (CBG) or combinations thereof.

In one embodiment, in the preceding composition, the cannabinoid is present in a concentration in the range of: CBC at 0.83% to 0.92% and CBG at 1.50% to 1.65%.

In one embodiment, in the preceding composition, the structural protein, one glycosaminoglycan, and mineral supplements are present in a concentration in the range of at 1.40% to 1.60%, 0.1% to 0.4% and 0.3% to 0.4%, respectively.

In one embodiment, in the preceding composition, the stabilizing/solubilizing agent is present in a concentration in the range of 1%-2%.

In one embodiment, in the preceding composition, the active principle is incorporated into a nanoplatform formulation comprising nanoplatform in the intra-articular injection.

In one embodiment, in the preceding composition, the stabilizing/solubilizing agents are medium chain triglycerides (MCT).

In one embodiment, in the preceding composition, the nanoplatform has an average particle size of less than 200 nm, and is formulated to ensure the controlled release of cannabinoids and other active ingredients over a sustained period.

In one embodiment, in the preceding composition, the formulation is suitable for intra-articular injection.

In another embodiment of the disclosure, the disclosure encompasses a method for treating osteoarthritis in a human individual, comprising: administering an effective concentration of the composition of Claim 1, wherein the active principle comprises a cannabinoid, two structural protein and calcium supplements, and the stabilizing solubilizing agent.

In one embodiment, in the preceding method, wherein the human individual is selected for treatment based on osteoarthritis diagnosis.

In one embodiment, in the preceding method, the individual is selected for treatment of: primary and secondary osteoarthritis.

In one embodiment, in the preceding method, further comprises identifying individuals using: medical history, physical examination, symptom diary, and additional tests.

In one embodiment, in the preceding method, the individual is identified by having alterations in: joint pain, stiffness, loss of mobility, inflammation, sensation of friction, muscle weakness, and joint deformity.

In one embodiment, in the preceding method, the effective concentration is administered as a single injection containing 70 mg of cannabinoids, 50 mg structural protein, and 10 mg calcium gluconate active for a period of every 3 months for 1 year.

In one embodiment, in the preceding method, the administering of the effective concentration of the composition acts as a monotherapy for treating Osteoarthritis.

In one embodiment, in the preceding method, the treatment composition alleviates: joint pain, stiffness, loss of mobility, inflammation, sensation of friction, muscle weakness, and joint deformity.

In another embodiment of the disclosure, the disclosure encompasses a method describes the production of a pharmaceutical composition for treating osteoarthritis of Claim 1, which combines cannabinoids, a structural protein, and a calcium supplement in a nanoplatform formulation with a stabilizing and solubilizing agent. The formulation is specifically designed for intra-articular delivery and is prepared using nano emulsification techniques, resulting in a nanoplatform with a size of less than 200 nm and sterilization through membrane filtration.

In another embodiment of the disclosure, the disclosure encompasses a composition for the treatment of osteoarthritis, comprising:
Cannabichromene (CBC) in a concentration ranging from 0.5% to 2%;
Cannabigerol (CBG) in a concentration ranging from 1% to 2.5%;
Glycosaminoglycans in a concentration ranging from 0.5% to 3%;
Structural proteins in a concentration ranging from 0.05% to 0.5%;
Mineral supplements in a concentration ranging from 0.05% to 2%; wherein the composition is formulated for intra-articular administration to relieve symptoms associated with osteoarthritis, including joint pain and cartilage degradation.

In another embodiment of the disclosure, the disclosure encompasses a composition for the treatment of osteoarthritis, comprising:
Cannabichromene (CBC) in a concentration ranging from 0.83% to 0.92%;
Cannabigerol (CBG) in a concentration ranging from 1.5% to 1.65%;
Hyaluronic acid in a concentration of 1.5%;
Collagen in a concentration of 0.17%;
Calcium gluconate in a concentration of 0.33%; wherein the composition is formulated for intra-articular administration to alleviate osteoarthritis symptoms by promoting joint lubrication, reducing inflammation, and supporting cartilage regeneration.

1. A composition for treatment of osteoarthritis, comprising:
a) an active principle comprising at least two cannabinoid;
b) an second active principle comprising at least one or more additional active agents including structural proteins, glycosaminoglycans, mineral supplement, or combinations thereof; and
optionally c) a stabilizing/solubilizing agent;
wherein the composition is suitable for intra articular administration.

2. The composition according to claim 1, wherein the cannabinoid comprises one or more of the following: cannabichromene (CBC), cannabigerol (CBG) or combinations thereof.

3. The composition according to claim 1, wherein the cannabinoid is present in a concentration in the range of: CBC at 0.83% to 0.92% and CBG at 1.50% to 1.65%.

4. The composition according to claim 1, wherein the structural protein, one glycosaminoglycan, and mineral supplements are present in a concentration in the range of at 1.40% to 1.60%, 0.1% to 0.4% and 0.3% to 0.4%, respectively.

5. The composition according to claim 1, wherein the stabilizing/solubilizing agent is present in a concentration in the range of 1%-2%.

6. The composition according to claim 1, wherein the active principle is incorporated into a nanoplatform formulation comprising nanoplatform in the intra-articular injection.

7. The composition according to claim 1, wherein the stabilizing/solubilizing agents are medium chain triglycerides (MCT).

8. The composition described in claim 1, wherein the nanoplatform has an average particle size of less than 200 nm, and is formulated to ensure the controlled release of cannabinoids and other active ingredients over a sustained period.

9. The composition according to claim 1, wherein the formulation is suitable for intra-articular injection.

10. A method for treating osteoarthritis in a human individual, comprising: administering an effective concentration of the composition of claim 1, wherein the active principle comprises a cannabinoid, two structural protein and calcium supplements, and the stabilizing solubilizing agent.

11. The method according to claim 10, wherein the human individual is selected for treatment based on osteoarthritis diagnosis.

12. The method according to claim 11, wherein the individual is selected for treatment of: primary and secondary osteoarthritis.

13. The method of claim 12, further comprises identifying individuals using: medical history, physical examination, symptom diary, and additional tests.

14. The method of claim 12, wherein the individual is identified by having alterations in: joint pain, stiffness, loss of mobility, inflammation, sensation of friction, muscle weakness, and joint deformity.

15. The method according to claim 11, wherein the effective concentration is administered as a single injection containing 70 mg of cannabinoids, 50 mg structural protein, and 10 mg calcium gluconate active for a period of every 3 months for 1 year.

16. The method according to claim 11, wherein the administering of the effective concentration of the composition acts as a monotherapy for treating Osteoarthritis.

17. The method of claim 11, wherein the treatment composition alleviates: joint pain, stiffness, loss of mobility, inflammation, sensation of friction, muscle weakness, and joint deformity.

18. This method describes the production of a pharmaceutical composition for treating osteoarthritis of claim 1, which combines cannabinoids, a structural protein, and a calcium supplement in a nanoplatform formulation with a stabilizing and solubilizing agent. The formulation is specifically designed for intra-articular delivery and is prepared using nano emulsification techniques, resulting in a nanoplatform with a size of less than 200 nm and sterilization through membrane filtration.

19. A composition for the treatment of osteoarthritis, comprising:
Cannabichromene (CBC) in a concentration ranging from 0.5% to 2%;
Cannabigerol (CBG) in a concentration ranging from 1% to 2.5%;
Glycosaminoglycans in a concentration ranging from 0.5% to 3%;
Structural proteins in a concentration ranging from 0.05% to 0.5%;
Mineral supplements in a concentration ranging from 0.05% to 2%; wherein the composition is formulated for intra-articular administration to relieve symptoms associated with osteoarthritis, including joint pain and cartilage degradation.

20. A composition for the treatment of osteoarthritis, comprising:
Cannabichromene (CBC) in a concentration ranging from 0.83% to 0.92%;
Cannabigerol (CBG) in a concentration ranging from 1.5% to 1.65%;
Hyaluronic acid in a concentration of 1.5%;
Collagen in a concentration of 0.17%;
Calcium gluconate in a concentration of 0.33%; wherein the composition is formulated for intra-articular administration to alleviate osteoarthritis symptoms by promoting joint lubrication, reducing inflammation, and supporting cartilage regeneration.
